(19) United States
(12) Patent Application Publication
(10) Pub. No.: US 2026/0109965 A1
(43) Pub. Date: Apr. 23, 2026

(54) PURIFICATION OF NUCLEOTIDES BY MEANS OF A MIXED-MODE PURIFICATION SYSTEM

(71) Applicant: ModernaTX, Inc., Cambridge, MA (US)

(72) Inventors: Jennifer Beaudoin, Littleton, MA (US); Matthew J. Burak, Raynham, MA (US); Christopher Ladd Effio, Cambridge, MA (US); Haripriya Sharma, Cambridge, MA (US)

(21) Appl. No.: 19/115,842

(22) PCT Filed: Sep. 27, 2023
(86) PCT No.: PCT/US2023/033883

§ 371 Date: Mar. 27, 2025

Related U.S. Application Data
(60) Provisional application No. 63/411,028, filed on Sep. 28, 2022.

(51) Int. Cl. C12N 15/10; B01J 20/26

(52) U.S. Cl. CPC C12N 15/101; B01J 20/262; C12N 15/1017

(57) ABSTRACT

This disclosure relates to purification of nucleotides using mixed mode resins.

### CROSS-REFERENCE

This application claims priority to U.S. provisional application No. 63/411,028, filed Sep. 28, 2022, which is incorporated by reference herein in its entirety.

### TECHNICAL FIELD

This disclosure relates to purification of nucleotides.

### BACKGROUND

There is great interest in the field of therapeutics to be able to purify oligonucleotides, such as mRNA. Oligonucleotides, such as mRNA, can be encapsulated in lipid nanoparticles and delivered to a subject for treatment or prevention of various diseases or conditions. The production cost of oligonucleotides can be relatively high. Efficient methods for purifying oligonucleotides can mitigate the production costs.

### BRIEF SUMMARY

Provided herein is a method of purifying an oligonucleotide, or salt thereof, comprising:
collecting and combining one or more mixtures comprising the oligonucleotide, or a salt thereof, and one or more contaminants; and
removing the contaminants from the combined mixtures using a mixed-mode purification system.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture; and
filtering the first mixture to provide a second mixture.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture;
filtering the first mixture to provide a second mixture; and
filtering the second mixture to provide a third mixture.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash; and
filtering the first mixture to provide a second mixture, wherein the filtering the first mixture to provide a second mixture comprises first tangential flow filtration and a first buffer exchange.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash; and
filtering the first mixture to provide a second mixture, wherein the filtering the first mixture to provide a second mixture comprises first tangential flow filtration and a first buffer exchange.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

Also provide herein is a method of purifying an oligonucleotide, or salt thereof, comprising:
collecting and combining one or more mixtures comprising the oligonucleotide, or a salt thereof, and one or more contaminants:
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

Also provide herein is a method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

Also provided herein is an oligonucleotide purified according to a method provided herein.

### BRIEF DESCRIPTION OF THE DRAWINGS

FIG. 1a shows an exemplary schematic of the purification process.

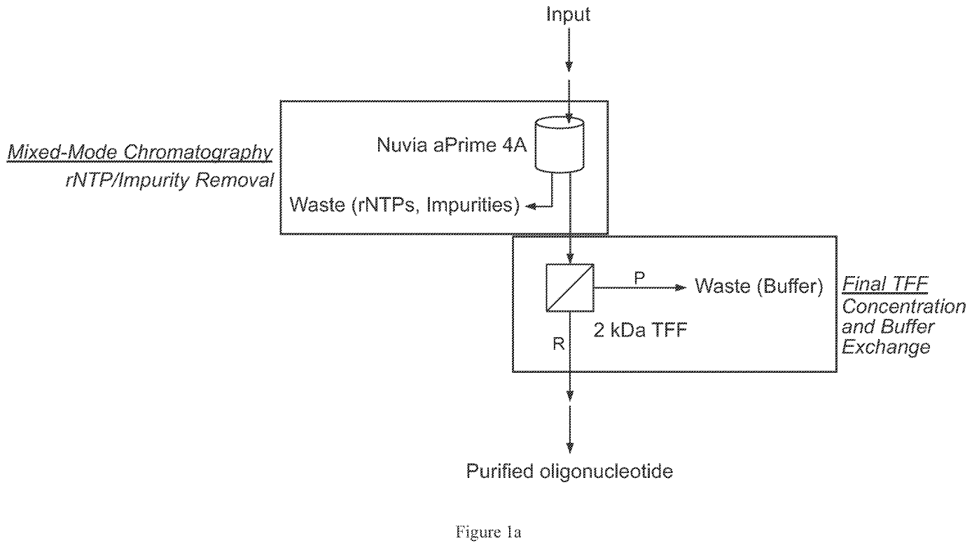

FIG. 1b shows an alternative exemplary schematic of the purification process.

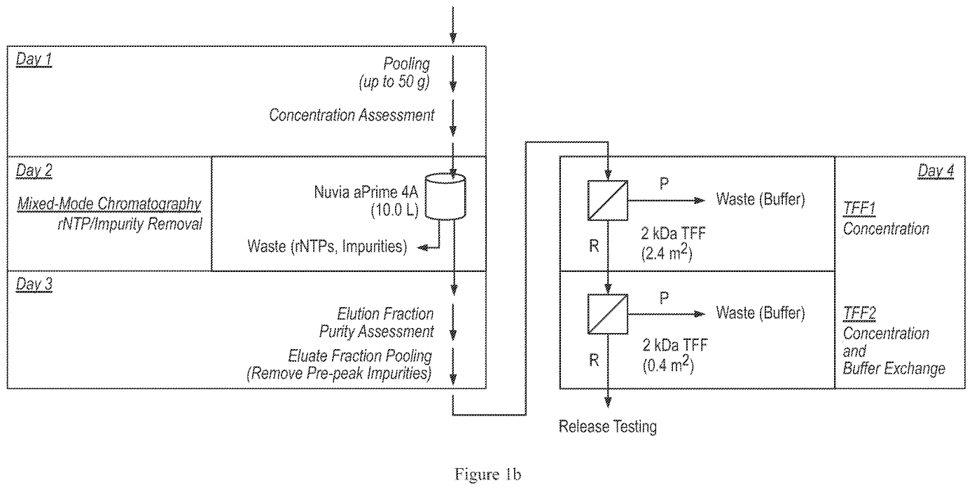

FIG. 2 shows absorbance at 260 nm of fractions collected from a mixed-mode chromatography system using TSKgel® SuperQ-5PW (30) (AEX) and Nuvia aPrime 4A (HIC/AEX) resins.

FIG. 3 shows absorbance at 260 nm of fractions collected from a mixed-mode chromatography system using a potassium phosphate wash step.

FIG. 4 shows absorbance at 260 nm of fractions collected from a mixed-mode chromatography system at high column volumes (CVs) of a potassium phosphate wash.

FIG. 5 shows absorbance at 260 nm of fractions collected from a mixed-mode chromatography system with different feed material and load challenges.

FIG. 6 shows sample purity at different elution volumes from a mixed-mode chromatography system.

FIG. 7 shows absorbance of fractions collected from a mixed-mode chromatography system using Nuvia aPrime 4A (HIC/AEX) resin with a 0.75 M NH₄Cl linear gradient wash.

FIG. 8 shows absorbance of fractions collected from a mixed-mode chromatography using Nuvia aPrime 4A (HIC/AEX) resin with a 0.75 M NH₄Cl wash.

FIG. 9 shows absorbance of fractions collected from a mixed-mode chromatography using Nuvia aPrime 4A (HIC/AEX) resin with a 0.75 M KCl linear gradient wash.

FIG. 10 shows absorbance of fractions collected from a mixed-mode chromatography using Nuvia aPrime 4A (HIC/AEX) resin with a 0.75 M NaCl linear gradient wash.

FIG. 11 shows the purity of Compound 4 collected from mixed-mode chromatography in different solutions.

FIG. 12 shows absorbance at of fractions collected from a mixed-mode chromatography system with a potassium phosphate wash followed by a potassium chloride elution.

FIG. 13 shows the purity of Compound 4 at different temperatures after mixed-mode chromatography using a potassium chloride wash.

FIG. 14 shows absorbance at of fractions collected from a mixed-mode chromatography system with a potassium phosphate wash followed by a potassium chloride elution on a 20-gram scale.

### DETAILED DESCRIPTION

Provided herein are methods of purifying oligonucleotides. The methods described herein provide procedures for collecting and combining the mixtures from, e.g., mRNA preparation, that contain oligonucleotides, and removing the contaminants to provide purified oligonucleotides. The methods described herein are efficient and can provide the oligonucleotides in high yields, e.g., greater than about 80%. In some instances, the yields can be greater than 90%. The purity of the oligonucleotides can be greater than 90%, greater than 98%, or greater than about 99%. In some instances, the purity of the oligonucleotides is greater than 99.5%.

In some embodiments, the method of purifying an oligonucleotide, or salt thereof, comprises:
collecting and combining one or more mixtures comprising the oligonucleotide, or a salt thereof, and one or more contaminants; and
removing the contaminants from the combined mixtures using a mixed-mode purification system.

In some embodiments, the oligonucleotide comprises 1 to 200, 1 to 175, 1 to 150, 1 to 125, 1 to 100, 1 to 75, 1 to 50, 1 to 25, 1 to 20, 1 to 15, 1 to 10, or 1 to 5 nucleotides. In some embodiments, the oligonucleotide comprises 1 to 5 nucleotides.

In some embodiments, the oligonucleotide comprises 3 or 4 nucleotides.

In some embodiments, the oligonucleotide, or a salt thereof, has a methylated guanosine and two or three nucleotides connected to a phosphate group.

In some embodiments, the oligonucleotide is an mRNA nucleotide cap.

In some embodiments, the oligonucleotide is a compound of Formula I:

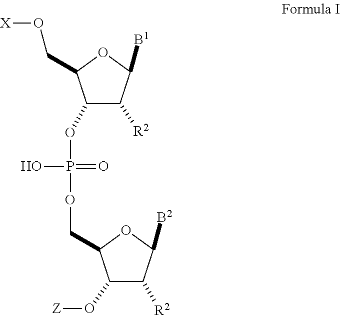

or salt thereof, wherein:
B¹ and B² are independently a natural, a modified, or an unnatural nucleoside based;
R¹ and R² are independently —OH or —OCH₃;
X is H or

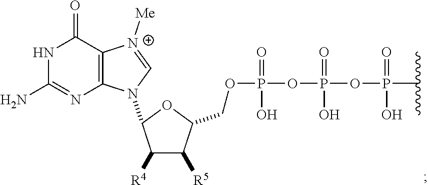

wherein R⁴ and R⁵ are independently —OH or —OCH₃; and
Z is H or

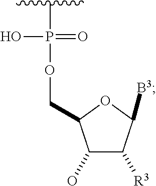

wherein B³ is a natural, a modified, or an unnatural nucleoside based; and
R³ is —OH or —OCH₃.

In some embodiments, X is H. In some embodiments, X is

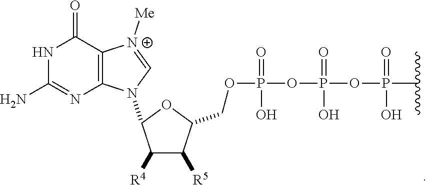

In some embodiments, Z is H. In some embodiments, Z is

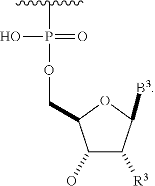

In some embodiments, X is H and Z is H. In some embodiments, X is H and Z is

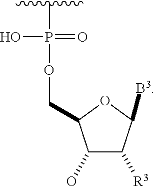

In some embodiments, X is

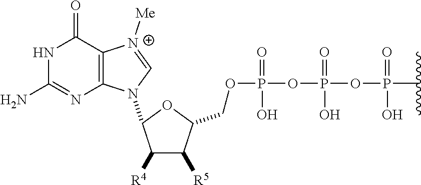

and Z is H. In some embodiments, X is

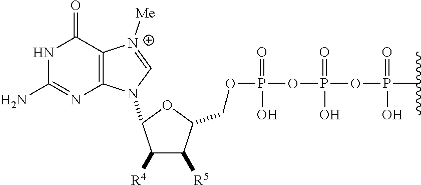

### and Z is

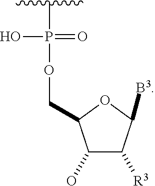

In some embodiments, B¹, B², and B³ are natural nucleoside bases. In some embodiments, at least one of B¹, B², and B³ is a modified or unnatural base. In some embodiments, at least one of B¹. B², and B³ is N6-methyladenine. In some embodiments, B₁ is adenine, cytosine, thymine, or uracil. In some embodiments, B¹ is adenine, B² is uracil, and B³ is adenine.

In some embodiments, B¹ and B² are natural nucleoside bases. In some embodiments, at least one of B¹ and B² is a modified or unnatural base. In some embodiments, at least one of B¹ and B² is N6-methyladenine. In some embodiments, B¹ is adenine, cytosine, thymine, or uracil. In some embodiments, B¹ is adenine and B² is uracil.

In some embodiments, R¹, R², and R³ are —OH. In some embodiments, R¹, R², and R³ are —OCH₃. In some embodiments, one of R¹, R², and R³ is —OH and the other two of R¹, R², and R³ are —OCH₃. In some embodiments, two of R¹, R², and R³ are —OH and the other one of R¹, R², and R³ is —OCH₃.

In some embodiments, R¹ and R² are —OH. In some embodiments, R¹ and R² are —OCH₃. In some embodiments. R¹ is —OH and R² is —OCH₃. In some embodiments, R¹ is —OCH₃ and R² is —OH.

In some embodiments, R⁴ and R⁵ are —OH. In some embodiments, R⁴ and R³ are —OCH₃. In some embodiments, R⁴ is —OH and R⁵ is —OCH₃. In some embodiments, R⁴ is —OCH₃ and R⁵ is —OH.

In some embodiments, the oligonucleotide is

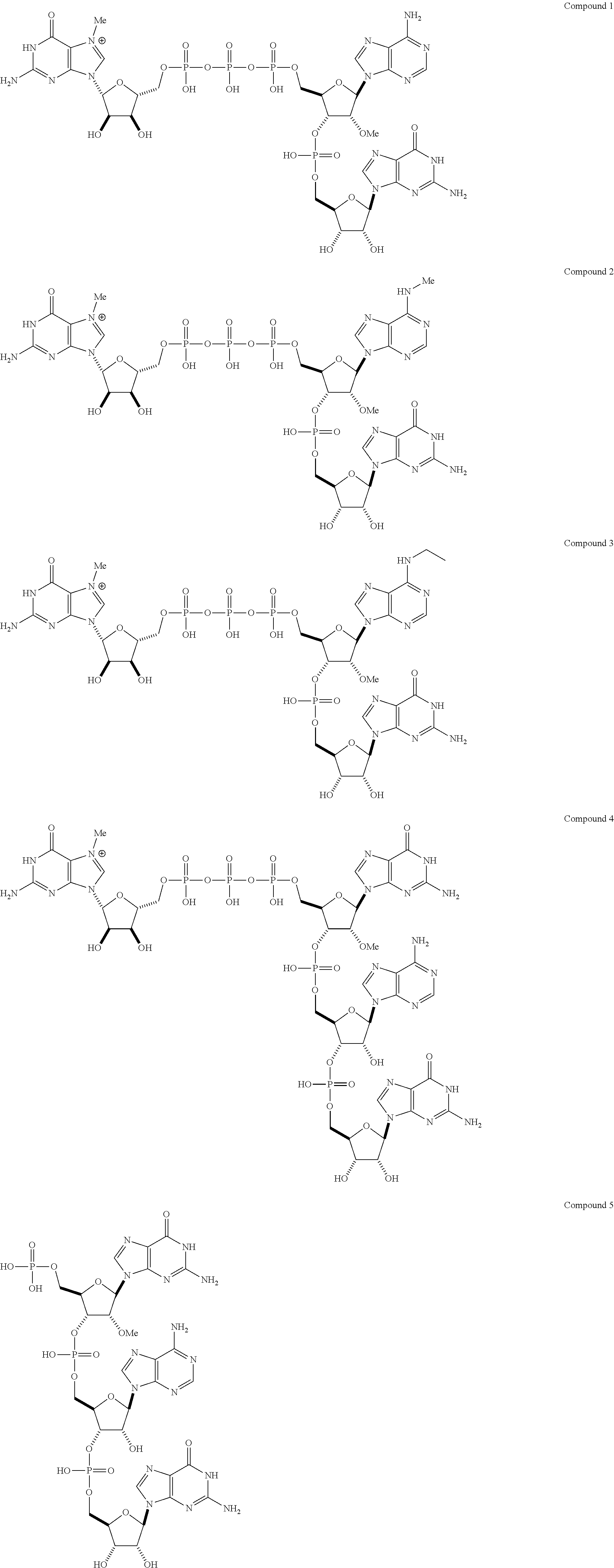

or a sat thereof.

In some embodiments, the oligonucleotide is a salt. For example, one or more protons of the phosphate groups or other acidic positions of the oligonucleotide can be deprotonated, generating an anionic oligonucleotide. In some embodiments, the cation of the anionic oligonucleotide is an alkali metal ion (e.g., Li⁺, Na⁺, K³⁰ , Cs⁺ etc.). In some embodiments, the cation is Na⁺. In some embodiments, the cation of the anionic oligonucleotide is a primary, secondary, tertiary ammonium, or quaternary ammonium cation. In some embodiments, the cation is a primary ammonium cation. In some embodiments, the cation is ammonium. In some embodiments, the cation is an alkyl primary ammonium cation. In some embodiments, the alkyl primary ammonium cation is R₁H₃N wherein R₁ is C₁₋₈ alkyl. In some embodiments, the alkyl primary ammonium cation is methylammonium. In some embodiments, the cation is a secondary ammonium cation. In some embodiments, the cation is an alkyl secondary ammonium cation. In some embodiments, the alkyl secondary ammonium cation is (R₁)₂H₂N wherein each R₁ is independently C₁₋₈ alkyl. In some embodiments, the alkyl secondary ammonium cation is dimethylammonium or methylethylammonium.

In some embodiments, the cation is a tertiary ammonium cation. In some embodiments, the cation is an alkyl tertiary ammonium cation. In some embodiments, the alkyl tertiary ammonium cation is (R₁)₃HN wherein each R₁ is independently C₁₋₈ alkyl. In some embodiments, the alkyl tertiary ammonium cation is dimethyloctylammonium, dimethylhexylammonium or triethylammonium. In some embodiments, the cation is a quaternary ammonium cation. In some embodiments, the cation is an alkyl quaternary ammonium cation. In some embodiments, the alkyl tertiary ammonium cation is (R₁)₄N wherein each R₁ is independently C₁₋₈ alkyl. In some embodiments, the alkyl quaternary ammonium cation is tetramethylammonium, trimethylethylammonium, or trimethylhexylammonium.

The anionic oligonucleotide can have one, two, three, four, five, or more negative charges. In some embodiments, the anionic oligonucleotide has one negative charge. In some embodiments, the anionic oligonucleotide has two negative charges.

In some embodiments, the anionic oligonucleotide has three negative charges. In some embodiments, the anionic oligonucleotide has four negative charges. The anionic oligonucleotide can have an average negative charge that is not limited to an integer, e.g., the average negative charge can be two and half, three and half, and four and half, etc. Salts of Compound 1 can include sodium salt (Na⁺), N,N-dimethyloctylammonium (DMOA) salt, dimethylhexylammonium (DMHA) salt, and primary ammonium (NH₄⁺) salt. Salts of Compound 2 can include sodium salt (Na⁺), DMOA salt, dimethylhexylammonium (DMHA) salt, and primary ammonium (NH₄⁺) salt. Salts of Compound 3 can include sodium salt (Na⁺), DMOA salt, dimethylhexylammonium (DMHA) salt, and primary ammonium (NH₄′) salt. Salts of Compound 4 can include sodium salt (Na⁺), DMOA salt, dimethylhexylammonium (DMHA) salt, and primary ammonium (NH₄⁺) salt. Salts of Compound 5 can include sodium salt (Na⁺), DMOA salt, dimethylhexylammonium (DMHA) salt, and primary ammonium (NH₄⁺) salt.

The cation associated with the oligonucleotide can change over the course of the purification process. For example, during the purification process the cation associated with the oligonucleotide can be exchanged to Na⁺, which is then exchanged to DMOA, which is then exchanged to NH₄⁺. In some embodiments, during the purification process the cation associated with the oligonucleotide is exchanged to Na′, which is then exchanged to DMHA, which is then exchanged to NH₄′. In some embodiments, during the purification process the cation associated with the oligonucleotide is exchanged to NH₄⁺, which is then exchanged to DMOA, which is then exchanged to NH₄⁺. In some embodiments, during the purification process the cation associated with the oligonucleotide is exchanged to NH₄⁺, which is then exchanged to DMHA, which is then exchanged to NH₄⁺. In some embodiments, during the purification process the cation associated with the oligonucleotide is exchanged to NH₄⁺, and the cation NH₄⁺ stays the same during the purification process.

In some embodiments, the oligonucleotide is

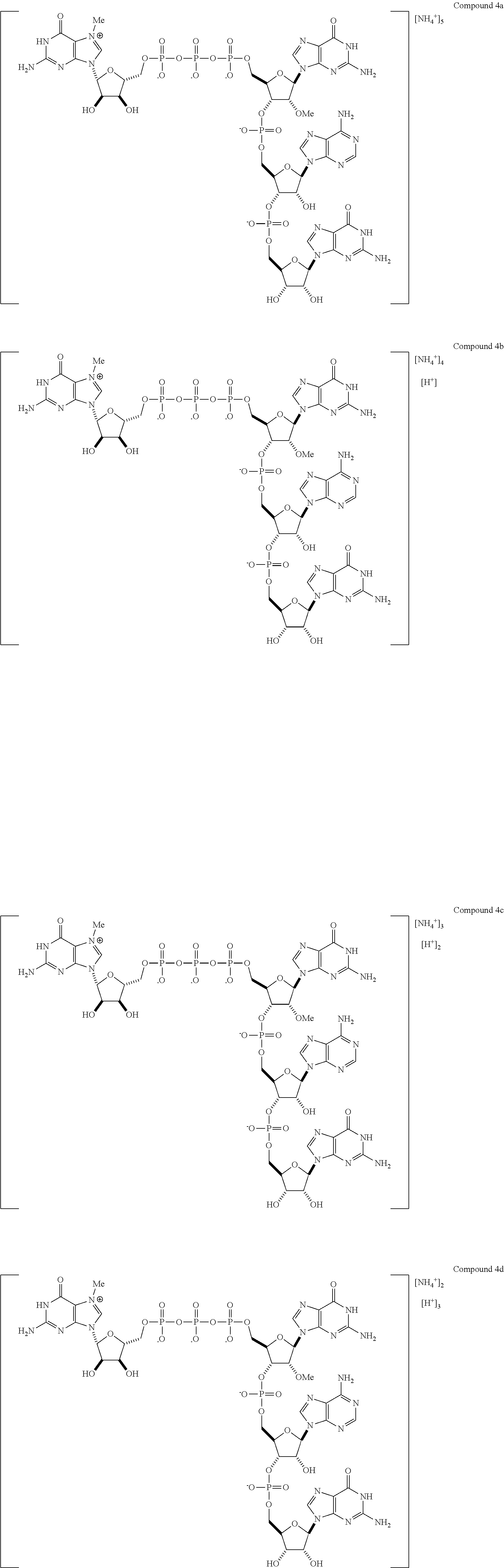

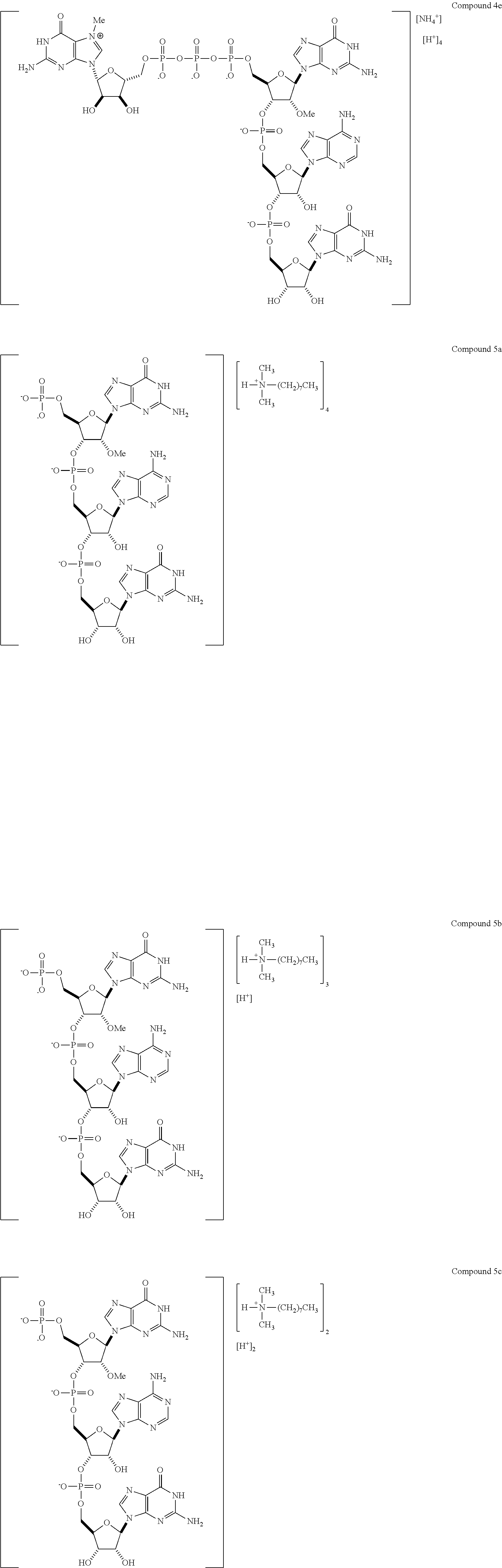

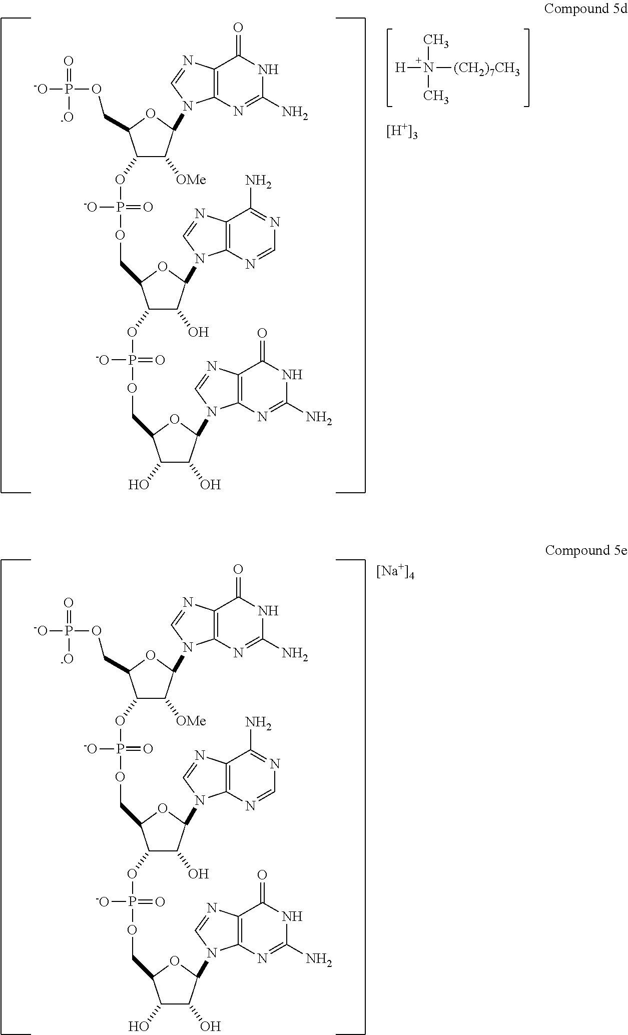

In some embodiments, the one or more mixtures that are collected and combined are from an mRNA preparation. In some embodiments, the mRNA preparation is an in vitro transcription preparation. In some embodiments, the one or more mixtures that are collected and combined from the purification of mRNA nucleotide caps that were prepared in a synthetic reaction mixtures. In some embodiments, the synthetic reaction mixture is a de novo preparation.

In some embodiments, the one or more contaminants comprise macromolecules, proteins, or combinations thereof. In some embodiments, the one or more contaminants comprise ribonucleoside triphosphates (rNTPs). In some embodiments, the rNTPs are rATP, rGTP, rCTP, rUTP, or m1Ψ, or combinations thereof. In some embodiments, the one or more contaminants comprise nucleic acid. In some embodiments, the nucleic acid is RNA or DNA. In some embodiments, the RNA is mRNA, tRNA, or rRNA.

In some embodiments, the method of purifying the oligonucleotide results in the oligonucleotide with a purity of about 70% to about 100%, about 80% to about 100%, about 80% to about 99%, about 85% to about 99%, about 90% to about 99%, or about 95% to about 99%.

In some embodiments, removing the contaminants from the combined mixtures using a mixed-mode purification system comprises passing the combined mixtures through a mixed-mode chromatography system to provide a first mixture. In some embodiments, the mixed-mode chromatography system comprises a mixed-mode resin. In some embodiments, the mixed-mode resin is a low ionic capacity resin.

In some embodiments, the mixed-mode resin is a high ionic capacity resin. In some embodiments, the mixed-mode resin comprises macroporous highly crosslinked polymer or high porosity cross-linked cellulose. In some embodiments, the mixed-mode resin comprises macroporous highly crosslinked polymer. In some embodiments, the mixed-mode resin comprises resins with hydrophobic and anion exchange properties. In some embodiments, the mixed-mode resin comprises a resin with hydrophobic and anion exchange properties. In some embodiments, the mixed-mode resin comprises an aromatic hydrophobic anion exchanger.

In some embodiments, the aromatic hydrophobic anion exchanger comprises a ligand of Formula II:

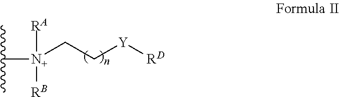

or a salt thereof, wherein:
   R^{A} is —CH₃, —CH₂CH₃, —(CH₂)₂CH₃, —CH(CH₃)₂, and —(CH₂)₃CH₃;
   R^{B} is —CH₃, —CH₂CH₃, —(CH₂)₂CH₃. —CH(CH₃)₂, and —(CH₂)₃CH₃;
   n is 0, 1, 2, 3, or 4;
   Y is absent, O, or NR^{C};
   R^{C} is H or —CH₃; and
   R^{D} is —CH₃, C₆₋₁₀ aryl, or 5 to 10 membered heteroaryl ring.

In some embodiments, R^{A} is —CH₃. In some embodiments, R^{A} is —CH₂CH₃. In some embodiments, R^{A} is —(CH₂)₂CH₃. In some embodiments, R^{A} is —CH(CH₃)₂. In some embodiments, R^{A} is —(CH₂)₃CH₃.

In some embodiments, R^{B} is —CH₃. In some embodiments, R^{B} is —CH₂CH₃. In some embodiments, R^{B} is —(CH₂)₂CH₃. In some embodiments, R^{B} is —CH(CH₃)₂. In some embodiments, R^{B} is —(CH₂)₃CH₃.

In some embodiments, n is 0. In some embodiments, n is 1. In some embodiments, n is 2. In some embodiments, n is 3. In some embodiments, n is 4.

In some embodiments, Y is absent. In some embodiments, Y is O. In some embodiments, Y is NR^{C}. In some embodiments, R^{C} is H. In some embodiments, R^{C} is —CH₃.

In some embodiments, R^{D} is —CH₃. In some embodiments, R^{D} IS C₆₋₁₀ aryl. In some embodiments, R^{D} is 5 to 10 membered heteroaryl ring.

In some embodiments, the ligand is:

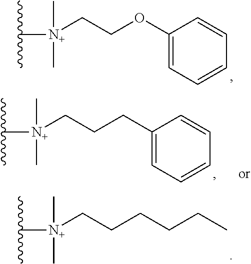

In some embodiments, the ligand is:

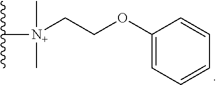 [SMILES: *[N+](C)(C)CCOc1ccccc1]

In some embodiments, the aromatic hydrophobic anion exchanger comprises a ligand having the formula:

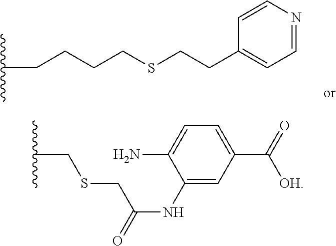 [SMILES: *CCCCSCCc1ccncc1 ; *CSCC(=O)Nc1cc(C(=O)O)ccc1N]

In some embodiments, the mixed-mode resin comprises a resin with hydrophobic, anion exchange, and hydrogen bonding properties.

In some embodiments, the resin with hydrophobic, anion exchange, and hydrogen bonding properties comprises a ligand having the formula:

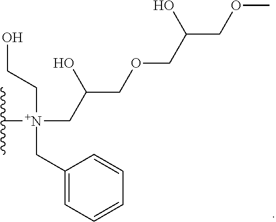 [SMILES: *[N+](CCO)(Cc1ccccc1)CC(O)COCC(O)COC]

In some embodiments, the density of the ligands is 100±20 μeq/ml. In some embodiments, the density of the ligands is (±20 μeq/ml) 50, 80, 100, 150, or 200 μeq/ml.

In some embodiments, the mixed-mode resin comprises a resin with calcium affinity and cation exchange priorities.

In some embodiments, the mixed-mode resin comprises particles with a median particle size of (±10 μm) 10 to 120, 20 to 100, 30 to 100, or 40 to 90 μm. In some embodiments, the mixed-mode resin comprises particles with a median particle size of (±10 μm) 40, 50, 75, or 90 μm. In some embodiments, the mixed-mode resin comprises particles with a median particle size of 50 z 10 μm.

In some embodiments, the mixed-mode resin is Capto™ Adhere, Capto™ Adhere ImpRes, HEA HyperCel, PPA HyperCelm Nuvia aPrime 4A, MEP HyperCel, CMM HyperCel, CHT Ceramic Hydroxyapatite XT Media, CHT Ceramic Hydroxyapatite and Bio-Gel® Crystalline Hydroxyapatite, MPC Ceramic Hydroxyfluoroapatite Resin, or CFT Ceramic Fluoroapatite Resin. In some embodiments, the mixed-mode resin is Capto™ Adhere, Capto™ Adhere ImpRes, HEA HyperCel™, PPA HyperCel™, or Nuvia aPrime 4A. In some embodiments, the mixed-mode resin is Nuvia aPrime 4A resin.

In some embodiments, the mixed-mode chromatography system comprises about 0.08 to about 1.0 g/L of resin, about 0.2 to about 0.8 g/L of resin, or about 0.4 to about 0.6 g/L of resin. In some embodiments, the mixed-mode chromatography system comprises about 0.4 to about 8 g/L of resin, about 0.6 to about 6 g/L of resin, or about 0.8 to about 4 g/L of resin. In some embodiments, the mixed-mode chromatography system comprises 0.9 to about 11 g/L of resin, about 1 to about 9 g/L of resin, or about 3 to about 7 g/L of resin. In some embodiments, the mixed-mode chromatography system comprises about 0.5 g/L of resin, about 2 g/L of resin, or about 5 g/L resin.

In some embodiments, the mixed-mode chromatography system comprises an about 3.5 to about 6.5 mL column, an about 4.0 to about 6.0 mL column, or an about 4.5 to about 5.5 mL column. In some embodiments, the mixed-mode chromatography system comprises an about 8.5 to about 11.5 mL column, an about 9.0 to about 11.0 mL column, or an about 9.5 to about 10.5 mL column. In some embodiments, the mixed-mode chromatography system comprises an about 5 mL column or an about 10 mL column.

In some embodiments, the mixed-mode chromatography system comprises a first wash. In some embodiments, the first wash is a first isocratic wash. In some embodiments, the first wash is a first linear gradient wash. In some embodiments, the first wash comprises a first wash solution. In some embodiments, the concentration of the first wash solution is about 0.1 M to about 0.7 M, about 0.2 M to about 0.6 M, or about 0.3 M to about 0.5 M. In some embodiments, the concentration of the first wash solution is about 0.4 M to about 1.6 M, about 0.6 M to about 1.4 M, or about 0.8 M to about 1.2 M. In some embodiments, the first wash solution has a pH of about 6.7 to about 7.3, about 6.8 to about 7.2, or about 6.9 to about 7.1. In some embodiments, the first wash solution comprises a phosphate salt solution. In some embodiments, the first wash solution comprises a potassium phosphate solution, an ammonium phosphate, or a sodium phosphate solution. In some embodiments, the first wash solution comprises a potassium phosphate solution. In some embodiments, the concentration of the potassium phosphate solution is about 0.4 M or about 1.0 M. In some embodiments, the potassium phosphate solution has a pH of about 6.7 to about 7.3, about 6.8 to about 7.2, or about 6.9 to about 7.1. In some embodiments, the potassium phosphate solution has a pH of about 7.0. In some embodiments, the first wash is run for about 7 to about 12 column volumes (CVs), about 8 to about 11 CVs. or about 9 to about 10 CVs. In some embodiments, the first wash is run for about 10 CVs.

In some embodiments, the first wash elutes bound contaminants. In some embodiments, the first wash elutes bound rNTP impurities. In some embodiments, the rNTPs are collected. In some embodiments, the rNTPs are isolated. In some embodiments, the rNTPs are isolated by filtration. In some embodiments, the rNTPs are isolated by tangential flow filtration. In some embodiments, the rNTPs are collected and isolated. In some embodiments, the first wash elutes bond rATP, rGTP, rCTP, rUTP, or m1Ψ, or combination thereof. In some embodiments, rATP is collected and isolated. In some embodiments, rGTP is collected and isolated. In some embodiments, rCTP is collected and isolated. In some embodiments, rUTP is collected and isolated. In some embodiments, m1T is collected and isolated.

In some embodiments, the mixed-mode chromatography system does not comprise a first wash.

In some embodiments, the mixed-mode chromatography system comprises a water wash. In some embodiments, the water wash comprises Milli-Qn water. In some embodiments, the first wash is run for about 3 to about 8 CVs, about 4 to about 7 CVs, or about 4 to about 6 CVs.

In some embodiments, the water wash is run for about 5 CVs.

In some embodiments, the mixed-mode chromatography system comprises a second wash. In some embodiments, the second wash is a second isocratic wash. In some embodiments, the second wash is a second linear gradient wash. In some embodiments, the second wash comprises a second wash solution. In some embodiments, the concentration of the second wash solution is about 0.1 M to about 0.7 M, about 0.2 M to about 0.6 M, or about 0.3 M to about 0.5 M. In some embodiments, the concentration of the second wash solution is about 0.4 M to about 1.6 M, about 0.6 M to about 1.4 M, or about 0.8 M to about 1.2 M. In some embodiments, the concentration of the second wash solution is about 0.25 M, about 0.5 M, about 0.75 M, or about 1.0 M. In some embodiments, the second wash solution has the same pH as the first wash solution. In some embodiments, the second wash solution has the same pH as the water wash. In some embodiments, the second wash solution has a pH of about 6.7 to about 7.3, about 6.8 to about 7.2, or about 6.9 to about 7.1.

In some embodiments, the second wash solution comprises a potassium chloride solution, a sodium sulfate solution, a sodium citrate solution, a sodium chloride solution, or an ammonium chloride solution. In some embodiments, the second wash solution comprises a potassium chloride solution. In some embodiments, the concentration of the potassium chloride solution is about 0.4 M or about 1.0 M. In some embodiments, the potassium chloride solution has a pH of about 6.7 to about 7.3, about 6.8 to about 7.2, or about 6.9 to about 7.1. In some embodiments, the potassium chloride solution has a pH of about 7.0.

In some embodiments, the second wash is run for about 8 to about 14 CVs, about 9 to about 13 CVs, or about 10 to about 12 CVs. In some embodiments, the second wash is run for about 10 CVs or about 12 CVs. In some embodiments, the second wash is run for about 17 to about 25 CVs, about 18 to about 23 CVs, or about 19 to about 21 CVs. In some embodiments, the second wash is run for about 20 CVs.

In some embodiments, the second wash elutes bound oligonucleotide.

In some embodiments, the mixed-mode chromatography system comprises a first isocratic wash and then a second isocratic wash. In some embodiments, the mixed-mode chromatography system comprises a first isocratic wash, then a water wash, and then a second isocratic wash. In some embodiments, the mixed-mode chromatography system comprises a first linear gradient wash and then a second linear gradient wash. In some embodiments, the mixed-mode chromatography system comprises a first linear gradient wash, then a water wash, and then a second linear gradient wash. In some embodiments, the mixed-mode chromatography system comprises a second linear gradient wash. In some embodiments, the mixed-mode chromatography system comprises a second isocratic gradient wash.

In some embodiments, the method further comprises filtering the first mixture to provide a second mixture. In some embodiments, the filtering the first mixture to provide a second mixture comprises a first tangential flow filtration. In some embodiments, the first tangential flow filtration comprises a cassette filter, a spiral wound filter, a hollow fiber filter, a tubular filter, or a flat plate filter. In some embodiments, the first tangential flow filtration comprises a cassette filter. In some embodiments, the first tangential flow filtration comprises a filter selected from a cellulose based membrane, a polyamide membrane, a polyethersulfone membrane, hydrophilic polyethersulfone membrane, polyvinylidene fluoride membrane, and a polyethylene membrane. In some embodiments, the first tangential flow filtration comprises a cellulose based membrane filter. In some embodiments, the first tangential flow filtration comprises a Sartocon® Slice Hydrosart® membrane, Sartocon® Hydrosart® membrane, Sartorius Hydrosart® membrane, or Hydrosart®) membrane. In some embodiments, the first tangential flow filtration comprises a filter having a total membrane area of about 2.1 to about 2.7 m², about 2.2 to about 2.6 m², or about 2.3 to about 2.5 m². In some embodiments, the first tangential flow filtration comprises a filter having a total membrane area of about 2.4 m². In some embodiments, the first tangential flow filtration comprises a filter having a total membrane area of about 0.1 to about 0.7 m2, about 0.2 to about 0.6 m², or about 0.3 to about 0.5 m². In some embodiments, the first tangential flow filtration comprises a filter having a total membrane area of about 0.4 m². In some embodiments, the first tangential flow filtration comprises a filter having a total membrane area of about 0.6 to about 1.8 m², about 0.8 to about 1.6 m², or about 1.0 to about 1.4 m². In some embodiments, the first tangential flow filtration comprises a filter having a total membrane area of about 1.2 m². In some embodiments, the first tangential flow filtration comprises a filter having a total membrane area of about 0.05 to about 0.7 m², about 0.07 to about 0.5 m², or about 0.09 to about 0.3 m². In some embodiments, the first tangential flow filtration comprises a filter having a total membrane area of about 0.1 m². In some embodiments, the first tangential flow filtration comprises a filter having a total membrane area of about 0.03 to about 0.09 m², about 0.04 to about 0.08 m², or about 0.05 to about 0.07 m². In some embodiments, the first tangential flow filtration comprises a filter having a total membrane area of about 0.06 m². In some embodiments, the first tangential flow filtration comprises a filter having a molecular weight cut off of about 50 Da to about 5 kDa, about 100 Da to about 2 kDa, or about 250 Da to about 2 kDa. In some embodiments, the first tangential flow filtration comprises a filter having a molecular weight cut off of about 2 kDa. In some embodiments, the filtering the first mixture to provide a second mixture adjusts the concentration of the first mixture to about 14 mM to about 22 mM, about 16 mM to about 24 mM, or about 18 mM to about 22 mM. In some embodiments, the filtering the first mixture to provide a second mixture adjusts the concentration of the first mixture to about 19 mM, about 20 mM, or about 21 mM.

In some embodiments, the filtering the first mixture to provide a second mixture comprises a first buffer exchange. In some embodiments, the first buffer exchange comprises exchanging the solution of the first mixture to water for injection. In some embodiments, the filtering the first mixture to provide a second mixture does not comprise a first buffer exchange.

In some embodiments, the method further comprises filtering the second mixture to provide a third mixture. In some embodiments, the filtering the second mixture to provide a third mixture comprises a second tangential flow filtration. In some embodiments, the second tangential flow filtration comprises a cassette filter, a spiral wound filter, a hollow fiber filter, a tubular filter, or a flat plate filter. In some embodiments, the second tangential flow filtration comprises a cassette filter. In some embodiments, the second tangential flow filtration comprises a filter selected from a cellulose based membrane, a polyamide membrane, a polyethersulfone membrane, hydrophilic polyethersulfone membrane, polyvinylidene fluoride membrane, and a polyethylene membrane. In some embodiments, the second tangential flow filtration comprises a cellulose based membrane filter. In some embodiments, the second tangential flow filtration comprises a Sartocon® Slice Hydrosart® membrane, Sartocon® Hydrosart® membrane, Sartorius Hydrosart® membrane, or Hydrosart® membrane. In some embodiments, the second tangential flow filtration comprises a filter having a total membrane area of about 2.1 to about 2.7 m², about 2.2 to about 2.6 m², or about 2.3 to about 2.5 m². In some embodiments, the second tangential flow filtration comprises a filter having a total membrane area of about 2.4 m². In some embodiments, the second tangential flow filtration comprises a filter having a total membrane area of about 0.1 to about 0.7 m², about 0.2 to about 0.6 m², or about 0.3 to about 0.5 m². In some embodiments, the second tangential flow filtration comprises a filter having a total membrane area of about 0.4 m². In some embodiments, the second tangential flow filtration comprises a filter having a total membrane area of about 0.6 to about 1.8 m², about 0.8 to about 1.6 m², or about 1.0 to about 1.4 m². In some embodiments, the second tangential flow filtration comprises a filter having a total membrane area of about 1.2 m². In some embodiments, the second tangential flow filtration comprises a filter having a total membrane area of about 0.05 to about 0.7 m², about 0.07 to about 0.5 m², or about 0.09 to about 0.3 m². In some embodiments, the second tangential flow filtration comprises a filter having a total membrane area of about 0.1 m². In some embodiments, the second tangential flow filtration comprises a filter having a total membrane area of about 0.03 to about 0.09 m², about 0.04 to about 0.08 m², or about 0.05 to about 0.07 m². In some embodiments, the second tangential flow filtration comprises a filter having a total membrane area of about 0.06 m². In some embodiments, the second tangential flow filtration comprises a filter having a molecular weight cut off of about 50 Da to about 5 kDa, about 100 Da to about 2 kDa, or 250 Da to about 2 kDa. In some embodiments, the second tangential flow filtration comprises a filter having a molecular weight cut off of about 2 kDa. In some embodiments, the filtering the second mixture to provide a third mixture adjusts the concentration of the second mixture to about 14 mM to about 22 mM, about 16 mM to about 24 mM, or about 18 mM to about 22 mM. In some embodiments, the filtering the second mixture to provide a third mixture adjusts the concentration of the second mixture to about 19 mM, about 20 mM, or about 21 mM.

In some embodiments, the filtering the second mixture to provide a third mixture the second mixture to provide a third mixture comprises a second buffer exchange. In some embodiments, the second buffer exchange comprises exchanging the solution of the second mixture to water for injection. In some embodiments, the filtering the second mixture to provide a third mixture does not comprise a second buffer exchange.

In some embodiments, the method further comprises isolating rNTPs from the contaminants. In some embodiments, the rNTPs are isolated by filtering the contaminants eluted from the first wash. In some embodiments, filtering the contaminants eluted from the first wash comprises a third tangential flow filtration. In some embodiments, the third tangential flow filtration comprises a cassette filter, a spiral wound filter, a hollow fiber filter, a tubular filter, or a flat plate filter. In some embodiments, the third tangential flow filtration comprises a cassette filter. In some embodiments, the third tangential flow filtration comprises a filter selected from a cellulose based membrane, a polyamide membrane, a polyethersulfone membrane, hydrophilic polyethersulfone membrane, polyvinylidene fluoride membrane, and a polyethylene membrane. In some embodiments, the third tangential flow filtration comprises a cellulose based membrane filter. In some embodiments, the third tangential flow filtration comprises a Sartocon® Slice Hydrosart® membrane, Sartocon® Hydrosart® membrane, Sartorius Hyydrosart® membrane, or Hydrosart® membrane. In some embodiments, the third tangential flow filtration comprises a filter having a molecular weight cut off of about 50 Da to about 5 kDa, about 100 Da to about 2 kDa. or about 250 Da to about 2 kDa. In some embodiments, the third tangential flow filtration comprises a filter having a molecular weight cut off of about 50 Da to about 100 Da.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture; and
filtering the first mixture to provide a second mixture.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture;
filtering the first mixture to provide a second mixture; and
filtering the second mixture to provide a third mixture.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash; and
filtering the first mixture to provide a second mixture, wherein the filtering the first mixture to provide a second mixture comprises first tangential flow filtration and a first buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash; and
filtering the first mixture to provide a second mixture, wherein the filtering the first mixture to provide a second mixture comprises first tangential flow filtration and a first buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

In some embodiments, the method of purifying an oligonucleotide, or salt thereof, comprises:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

Also provided herein is an oligonucleotide purified according to a method provided herein.

In order that the present disclosure can be more readily understood, certain terms are first defined. As used in this application, except as otherwise expressly provided herein, each of the following terms shall have the meaning set forth below. Additional definitions are set forth throughout the application.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Alkyl: As used herein, the term “alkyl”, employed alone or in combination with other terms, refers to a saturated hydrocarbon group that may be straight-chain or branched. In some embodiments, the alkyl group contains 1 to 12, 1 to 8, or 1 to 6 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like. In some embodiments, the alkyl moiety is methyl, ethyl, n-propyl, isopropyl, n-butyl, isobutyl, tert-butyl, n-pentyl, isopentyl, neopentyl, n-hexyl, or 2,4,4-trimethylpentyl. In some embodiments, the alkyl moiety is methyl.

About: The term “about” as used in connection with a numerical value throughout the specification and the claims denotes an interval of accuracy, familiar and acceptable to a person skilled in the art. Such interval of accuracy is ±10%.

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

De Novo: As used herein, the term “de novo” refers to the synthesis of oligonucleotides from nucleosides.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

Isolated: As used herein, the term “isolated” refers to a substance or entity that has been separated from at least some of the components with which it was associated (whether in nature or in an experimental setting). Isolated substances (e.g., compounds) can have varying levels of purity in reference to the substances from which they have been isolated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

In some embodiments, the compounds described herein, and salts thereof, are substantially isolated. Methods for isolating compounds and their salts are routine in the art.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

Purified: As used herein, “purify,” “purified,” “purification” means to make substantially pure or clear from unwanted components, material defilement, admixture or imperfection.

Salts: The term “salt” includes any anionic and cationic complex. Salts can include pharmaceutically acceptable salts. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Stereoisomer: As used herein, the term “stereoisomer” refers to all possible different isomeric as well as conformational forms that a compound can possess (e.g., a compound of any formula described herein), in particular all possible stereochemically and conformationally isomeric forms, all diastereomers, enantiomers and/or conformers of the basic molecular structure. Some compounds of the present disclosure can exist in different tautomeric forms, all of the latter being included within the scope of the present disclosure.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

The methods described herein can be monitored according to any suitable method known in the art. For example, product formation can be monitored by spectroscopic means, such as nuclear magnetic resonance spectroscopy (e.g., ¹H or ¹³C), infrared spectroscopy, or spectrophotometry (e.g., UV-visible); or by chromatography such as high performance liquid chromatography (HPLC), liquid chromatography-mass spectrometry (LCMS or LC-MS), or thin layer chromatography (TLC) or other related techniques.

The methods described herein can be carried out in suitable solvents which can be readily selected by one of skill in the art of organic synthesis. Suitable solvents can be substantially nonreactive with the components (e.g., mRNA nucleotide caps) at the temperatures at which the processes are carried out, e.g., temperatures which can range from the solvent's freezing temperature to the solvent's boiling temperature. A given processes can be carried out in one solvent or a mixture of more than one solvent. Depending on the particular step, suitable solvents for a particular step can be selected. In some embodiments, methods described herein is to remove one or more solvents e.g., by heating, in vacuum such as rotary evaporator (rotovap).

Examples of solvents described herein can be an organic solvent, polar solvent, water, etc. or mixtures thereof. For example, the solvent can be a halogenated solvent, which can include carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

The solvent can be an organic solvent such as ether solvent, which can include dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof and the like.

The solvent can be an organic solvent such as a hydrocarbon solvent, which can include benzene, cyclohexane, pentane, hexane, toluene, cycloheptane, methylcyclohexane, heptane (e.g., n-heptane), ethylbenzene, in-, o-, or p-xylene, octane, indane, nonane, naphthalene, mixtures thereof, and the like.

The solvent can be a polar solvent, which can be protic or aprotic solvent. Examples of protic solvents can include water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, mixtures thereof, and the like.

Examples of aprotic solvents can include tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane. N,N-dimethylpropionamide, tetramethylurea, nitromethane, nitrobenzene, hexamethylphosphoramide, mixtures thereof, and the like.

In some embodiments, the compounds described herein, and salts thereof, can be found together with other substances such as water and solvents (e.g., hydrates and solvates).

The methods described herein can be carried out at appropriate temperatures, which can be readily determined by the skilled artisan. Temperatures will depend on, for example, the melting and boiling points of the components and solvent. “Elevated temperature” refers to temperatures above room temperature (about 22° C.). The expressions, “ambient temperature” and “room temperature” or “rt” as used herein, are understood in the art, and refer generally to a temperature, e.g., a reaction temperature, that is about the temperature of the room in which the method is carried out, for example, a temperature from about 20° C. to about 30° C.

The following examples are offered for illustrative purposes, and are not intended to be limiting. Those of skill in the art will readily recognize a variety of noncritical parameters, which can be changed or modified to yield essentially the same results.

Section and table headings are not intended to be limiting.

### EXAMPLES

Materials: TSKgel® SuperQ-5PW (20), TSKgel® SuperQ-5PW (30), TSKgel® SuperQ-5PW (20), and TSKgel® SuperQ-650S were purchased from Tosoh Bioscience. Nuvia aPrime 4A was purchased form Bio-Rad. POROS™ HQ, POROS™ XQ, and POROS™ XS were purchased from ThermoFisher. Capto™ DEAE, Capto™ Q, Capto™ Q ImpRes, Capto™ Adhere, and Capto™ Adhere ImpRes were purchased from Cytiva. HEA HyperCel™ and PPA HyperCel™ were purchased from Sartorius. Sartocon Slice Hydrosart®, Sartocon® Hydrosart®, Sartorius Hydrosart®, Hydrosart® were purchased from Sartorius. Milli-Q® water was obtained from a Millipore system.

### Example 1

### AEX Resin Screen

A mixture containing Compound 4 collected from an in vitro transcription (IVT) preparation was loaded (0.5 g/L of resin) onto columns packed with various anion exchange (AEX) resins including TSKgel® SuperQ-5PW (20), TSKgel® SuperQ-5PW (30), TSKpearl SuperQ-650S, Capto™ DEAE, Capto™ Q, Capto™ Q ImpRes, POROS™ HQ, and POROS™ XQ. After loading each column, the column was chased with Milli-Q®) water for 5 column volumes (CVs) and then washed with 20 mM Tris-HCl pH 7.5 for 5 CVs. The contents of the AEX columns were eluted using a 0 (20 mM Tris-HCl pH 7.5)—100% (20 mM Tris-HCl pH 7.5, 1 M ammonium chloride) linear gradient over 20 CVs (5% per CV).

Compound 4 (species 2) and rNTP (species 1) peak resolution (R) at the half-height peak width was then calculated as follows:

$$R = 1.18 \times \frac{{t}_{R 2} - {t}_{R 1}}{{W}_{0.5 h 2} - {W}_{0.5 h 1}} \qquad eq . ( 1 )$$

where t_{R} and W_{0.5h} were the retention time and half-height peak width, respectively.

TSKgel® SuperQ-5PW (20), TSKgel® SuperQ-5PW (30), TSKpearl SuperQ-650S. and POROS™ XQ resulted in the highest rNTP/Compound 4 peak resolution among all AEX resins tested (refer to Table 1.1).

TABLE 1.1
Anion Exchange Chromatography (AEX) resin,
column dimensions, and peak resolution.

| | Particle | Column | Column | Bed | |
|---|---|---|---|---|---|
| | Size | Volume | I.D. | Height | Resolution |
| Resin | (μm) | (mL) | (cm) | (cm) | (R) |
| TSKgel ® SuperQ- | 20 | 5.0 | 0.8 | 10.1 | 1.16 |
| 5PW (20) | | | | | |
| TSKgel ® SuperQ- | 30 | 5.0 | 0.8 | 10.1 | 1.01 |
| 5PW (30) | | | | | |
| TSKpearl SuperQ- | 35 | 5.0 | 0.8 | 10.1 | 1.00 |
| 650S | | | | | |
| POROS ™ HQ | 50 | 5.0 | 0.8 | 10.1 | 0.50 |
| POROS ™ XQ | 50 | 5.0 | 0.8 | 10.1 | 1.06 |
| Capto ™ DEAE | 90 | 5.0 | 1.6 | 2.5 | 0.79 |
| Capto ™ Q | 90 | 5.0 | 1.6 | 2.5 | 0.79 |
| Capto ™ Q ImpRes | 40 | 5.0 | 1.6 | 2.5 | 0.78 |

### Example 2

### Mixed-Mode Resin Screen

A mixture containing Compound 4 collected from an IVT preparation was loaded (0.5 g/L of resin) onto columns packed with various mixed-mode resins including Capto™ Adhere, Capto™ Adhere ImpRes, HEA HyperCel™, PPA HyperCel™, and Nuvia aPrime 4A. After loading each column, the columns were chased and washed with Milli-Q® water and 20 mM Tris-HCl pH 7.5 for 5 CVs. The contents of the columns were then eluted using a 0 (20 mM Tris-HCl pH 7.5)—100% (20 mM Tris-HCl pH 7.5, 1 M ammonium chloride) linear gradient over 20 CVs (5% per CV). Compound 4 and rNTP peak resolution at half-height peak width was then calculated according to eq (1).

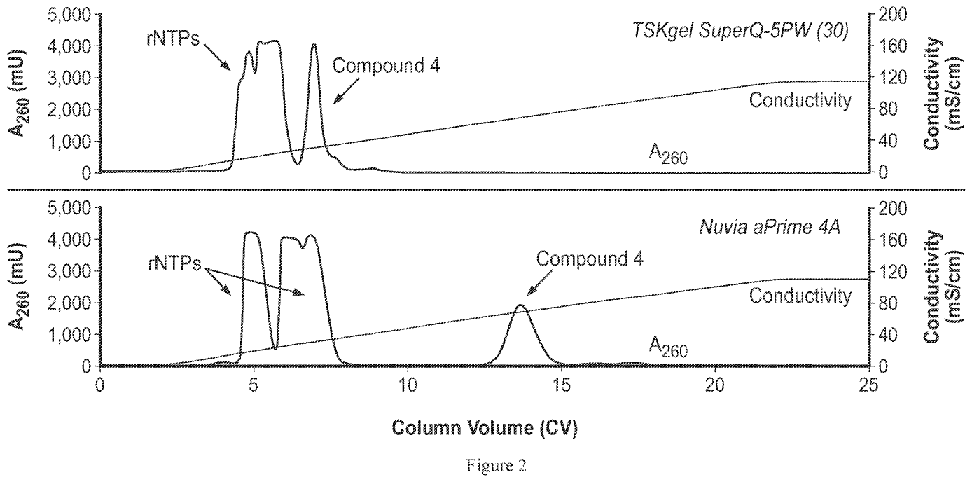

All mixed-mode resins tested resulted in higher rNTP/Compound 4 peak resolution than the peak resolution when AEX resins were used (see Table 2.1 and 2.2). Nuvia aPrime 4A provided the highest degree of separation between the two species (see FIG. 2).

TABLE 2.1
Anion Exchange Chromatography (AEX) resin,
column dimensions, and peak resolution.

| | Particle | Column | Column | Bed | |
|---|---|---|---|---|---|
| | Size | Volume | I.D. | Height | Resolution |
| Resin | (μm) | (mL) | (cm) | (cm) | (R) |
| TSKgel ® SuperQ- | 30 | 5.0 | 0.8 | 10.1 | 1.01 |
| 5PW (30) | | | | | |
| TSKpearl SuperQ- | 35 | 5.0 | 0.8 | 10.1 | 1.00 |
| 650S | | | | | |
| POROS ™ HQ | 50 | 5.0 | 0.8 | 10.1 | 0.50 |
| POROS ™ XQ | 50 | 5.0 | 0.8 | 10.1 | 1.06 |
| Capto ™ DEAE | 90 | 5.0 | 1.6 | 2.5 | 0.79 |
| Capto ™ Q | 90 | 5.0 | 1.6 | 2.5 | 0.79 |
| Capto ™ Q Impress | 40 | 5.0 | 1.6 | 2.5 | 0.78 |

TABLE 2.2
Mixed-Mode resin: Hydrophobic Interaction Chromatography
(HIC) and Anion Exchange Chromatography (AEX),
column dimensions, and peak resolution.

| | Particle | Column | Column | Bed | |
|---|---|---|---|---|---|
| | Size | Volume | I.D. | Height | Resolution |
| Resin | (μm) | (mL) | (cm) | (cm) | (R) |
| Capto ™ Adhere | 75 | 5.0 | 1.6 | 2.5 | 1.67 |
| Capto ™ Adhere | 40 | 5.0 | 1.6 | 2.5 | 2.02 |
| ImpRes | | | | | |
| HEA HyperCel ™ | 90 | 5.0 | 0.8 | 2.5 | 1.28 |
| PPA HyperCel ™ | 90 | 5.0 | 0.8 | 10.1 | 1.81 |
| Nuvia aPrime 4A | 50 | 5.0 | 0.8 | 10.1 | 2.47 |

### Example 3

### Resin and Salt Screen

Reaction crude containing Compound 5 sodium salt was loaded (0.5 g/L of resin) onto columns packed with various AEX and mixed-mode resins including TSKgel® SuperQ-5PW (20), TSKgel® SuperQ-5PW (30), TSKgel® SuperQ-650S, POROS™ XS, Capto™ Adhere ImpRes, Nuvia aPrime 4A. HEA HyperCel™, and PPA HyperCel™. After loading each column, the columns were chased with Milli-Q® water and then eluted with a 0-100% linear gradient of various salt buffers/solutions over 20 CVs. The salt buffers/solutions included ammonium chloride, sodium chloride, potassium chloride, potassium phosphate, sodium citrate, and sodium sulfate. Elution peaks were then collected (300-300 mAU) and analyzed using HPLC. Compound 5 purity and recovery from each resin and elution condition tested were determined.

Potassium phosphate (1 M; pH 7) provided the highest Compound 5 purity and recovery among all salt buffers/solutions for each AEX resin evaluated (see Table 3.1) However, it was not capable of eluting Compound 5 from mixed-mode resins such as Nuvia aPrime 4A resin (see Table 3.2).

TABLE 3.1
AEX resins, salt buffers/solutions, Compound 5 recovery and purity.

| | Particle | | | | | |
|---|---|---|---|---|---|---|
| | Size | | | Recovery | Purity | |
| Resin | (μm) | Cation | Anion | (R) | (P) | R × P |
| TSKgel ® | 20 | Ammonium | Chloride | 0.83 | 0.65 | 0.54 |
| SuperQ-5PW | | | | | | |
| (20) | | | | | | |
| TSKgel ® | 20 | Potassium | Chloride | 0.82 | 0.65 | 0.53 |
| SuperQ-5PW | | | | | | |
| (20) | | | | | | |
| TSKgel ® | 20 | Sodium | Chloride | 0.83 | 0.65 | 0.54 |
| SuperQ-5PW | | | | | | |
| (20) | | | | | | |
| TSKgel ® | 20 | Potassium | Phosphate* | 0.86 | 0.92 | 0.79 |
| SuperQ-5PW | | | | | | |
| (20) | | | | | | |
| TSKgel ® | 20 | Sodium | Sulfate | 0.86 | 0.75 | 0.64 |
| SuperQ-5PW | | | | | | |
| (20) | | | | | | |
| TSKgel ® | 20 | Sodium | Citrate** | 0.94 | 0.61 | 0.57 |
| SuperQ-5PW | | | | | | |
| (20) | | | | | | |
| TSKgel ® | 30 | Ammonium | Chloride | — | — | — |
| SuperQ-5PW | | | | | | |
| (30) | | | | | | |
| TSKgel ® | 30 | Potassium | Chloride | 0.84 | 0.62 | 0.52 |
| SuperQ-5PW | | | | | | |
| (30) | | | | | | |
| TSKgel ® | 30 | Sodium | Chloride | 0.82 | 0.61 | 0.50 |
| SuperQ-5PW | | | | | | |
| (30) | | | | | | |
| TSKgel ® | 30 | Potassium | Phosphate* | 0.85 | 0.85 | 0.73 |
| SuperQ-5PW | | | | | | |
| (30) | | | | | | |
| TSKgel ® | 30 | Sodium | Sulfate | — | — | — |
| SuperQ-5PW | | | | | | |
| (30) | | | | | | |
| TSKgel ® | 30 | Sodium | Citrate** | — | — | — |
| SuperQ-5PW | | | | | | |
| (30) | | | | | | |
| TSKgel ® | 35 | Ammonium | Chloride | — | — | — |
| SuperQ-650S | | | | | | |
| TSKgel ® | 35 | Potassium | Chloride | 0.84 | 0.70 | 0.58 |
| SuperQ-650S | | | | | | |
| TSKgel ® | 35 | Sodium | Chloride | 0.82 | 0.55 | 0.45 |
| SuperQ-650S | | | | | | |
| TSKgel ® | 35 | Potassium | Phosphate* | 0.87 | 0.83 | 0.72 |
| SuperQ-650S | | | | | | |
| TSKgel ® | 35 | Sodium | Sulfate | — | — | — |
| SuperQ-650S | | | | | | |
| TSKgel ® | 35 | Sodium | Citrate** | — | — | — |
| SuperQ-650S | | | | | | |
| POROS ™ XS | 50 | Ammonium | Chloride | — | — | — |
| POROS ™ XS | 50 | Potassium | Chloride | 0.84 | 0.62 | 0.52 |
| POROS ™ XS | 50 | Sodium | Chloride | 0.82 | 0.65 | 0.53 |
| POROS ™ XS | 50 | Potassium | Phosphate* | 0.86 | 0.69 | 0.59 |
| POROS ™ XS | 50 | Sodium | Sulfate | 0.86 | 0.64 | 0.55 |
| POROS ™ XS | 50 | Sodium | Citrate** | 0.88 | 0.58 | 0.51 |

All buffers/solutions were prepared at 1M
*1M Potassium Phosphate pH 7.0
**1M Sodium Citrate pH 6.5

TABLE 3.2
Mixed-mode (HIC/AEX) resins, salt buffers/solutions,
Compound 5 recovery and purity.

| | Particle | | | | | |
|---|---|---|---|---|---|---|
| | Size | | | Recovery | Purity | |
| Resin | (μm) | Cation | Anion | (R) | (P) | R × P |
| Capto ™ | 40 | Ammonium | Chloride | 0.82 | 0.83 | 0.68 |
| Adhere | | | | | | |
| ImpRes | | | | | | |
| Capto ™ | 40 | Potassium | Chloride | 0.80 | 0.81 | 0.66 |
| Adhere | | | | | | |
| ImpRes | | | | | | |
| Capto ™ | 40 | Sodium | Chloride | 0.82 | 0.83 | 0.68 |
| Adhere | | | | | | |
| ImpRes | | | | | | |
| Capto ™ | 40 | Potassium | Phosphate* | Did Not Elute | | |
| Adhere | | | | | | |
| ImpRes | | | | | | |
| Capto ™ | 40 | Sodium | Sulfate | Did Not Elute | | |
| Adhere | | | | | | |
| ImpRes | | | | | | |
| Capto ™ | 40 | Sodium | Citrate** | 0.84 | 0.89 | 0.75 |
| Adhere | | | | | | |
| ImpRes | | | | | | |
| Nuvia aPrime | 50 | Ammonium | Chloride | 0.86 | 0.66 | 0.57 |
| 4A | | | | | | |
| Nuvia aPrime | 50 | Potassium | Chloride | 0.88 | 0.66 | 0.58 |
| 4A | | | | | | |
| Nuvia aPrime | 50 | Sodium | Chloride | 0.82 | 0.67 | 0.55 |
| 4A | | | | | | |
| Nuvia aPrime | 50 | Potassium | Phosphate* | Did Not Elute | | |
| 4A | | | | | | |
| Nuvia aPrime | 50 | Sodium | Sulfate | 0.53 | 0.90 | 0.47 |
| 4A | | | | | | |
| Nuvia aPrime | 50 | Sodium | Citrate** | 0.86 | 0.85 | 0.73 |
| 4A | | | | | | |
| HEA | 90 | Ammonium | Chloride | — | — | — |
| HyperCel ™ | | | | | | |
| HEA | 90 | Potassium | Chloride | — | — | — |
| HyperCel ™ | | | | | | |
| HEA | 90 | Sodium | Chloride | 0.85 | 0.76 | 0.65 |
| HyperCel ™ | | | | | | |
| HEA | 90 | Potassium | Phosphate* | — | — | — |
| HyperCel ™ | | | | | | |
| HEA | 90 | Sodium | Sulfate | — | — | — |
| HyperCel ™ | | | | | | |
| HEA | 90 | Sodium | Citrate** | — | — | — |
| HyperCel ™ | | | | | | |
| PPA | 90 | Ammonium | Chloride | — | — | — |
| HyperCel ™ | | | | | | |
| PPA | 90 | Potassium | Chloride | — | — | — |
| HyperCel ™ | | | | | | |
| PPA | 90 | Sodium | Chloride | 0.83 | 0.79 | 0.66 |
| HyperCel ™ | | | | | | |
| PPA | 90 | Potassium | Phosphate* | — | — | — |
| HyperCel ™ | | | | | | |
| PPA | 90 | Sodium | Sulfate | — | — | — |
| HyperCel ™ | | | | | | |
| PPA | 90 | Sodium | Citrate** | — | — | — |
| HyperCel ™ | | | | | | |

All buffers/solutions were prepared at 1M
*1M Potassium Phosphate pH 7.0
**1M Sodium Citrate pH 6.5

### Example 4

### Evaluating the Use of a Potassium Phosphate Wash Step

A mixture containing Compound 4 collected from an IVT preparation was loaded (0.5 g/L of Nuvia aPrime 4A) onto a 5 mL column (0.8×10.0 cm). Columns were washed with either Milli-Q® water or a linear gradient comprised of 0-100% 1 M potassium phosphate pH 7.0 for 20 CVs. Columns were then washed for an additional 5 CVs with Milli-QR water. The remaining unbound material was eluted from the columns using a linear gradient of 0-100% potassium chloride.

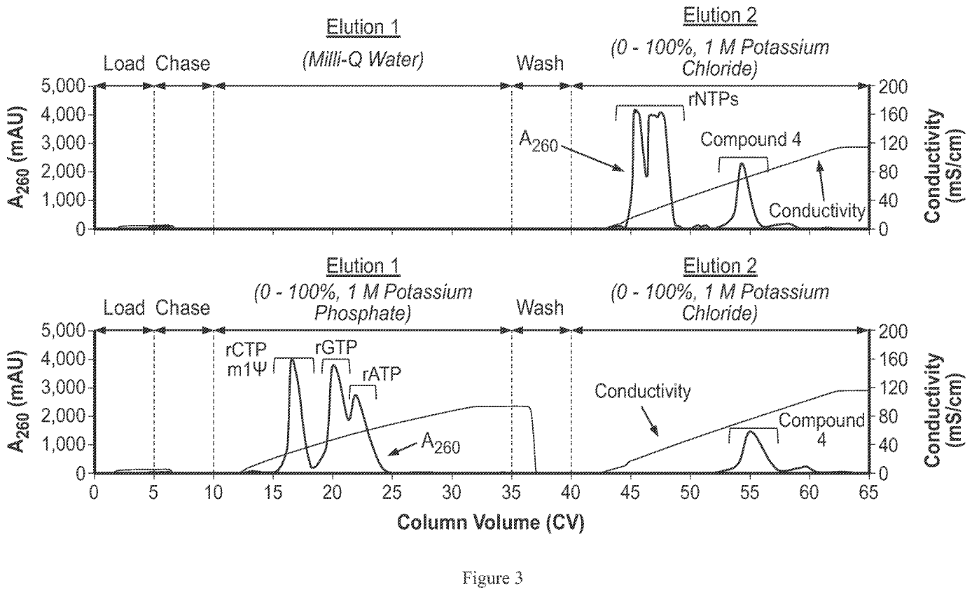

Potassium phosphate can remove bound rNTPs without impacting Compound 4 binding or inducing substantial peak shifting (see FIG. 3). Additionally, between 300-500 mM potassium phosphate pH 7.0 is required to remove rNTPs.

### Example 5

### Determining the Potassium Phosphate Wash Volume

A mixture containing Compound 4 collected from an IVT preparation was loaded (2 or 5 g/L of resin) onto a 10 mL (1.0×12.7 cm) column packed with low ionic capacity Nuvia aPrime 4A resin (87 μeq/mL) in order to simulate the column bed height and geometry at scale. Additionally, a pump wash was performed prior to sample application in order to prime the system with load material and reduce the delay volume. After sample application on the columns, the columns were washed with 0.4 M potassium phosphate pH 7.0 for 50 CVs. Columns were then washed with Milli-QR water for 5 CVs. Finally, the columns were cleaned with 1 M NaOH for 5 CVs, neutralized in 1 M Tris-HCl pH 7.5 for 5 CVs. and rinsed in Milli-QG water for 5 CVs.

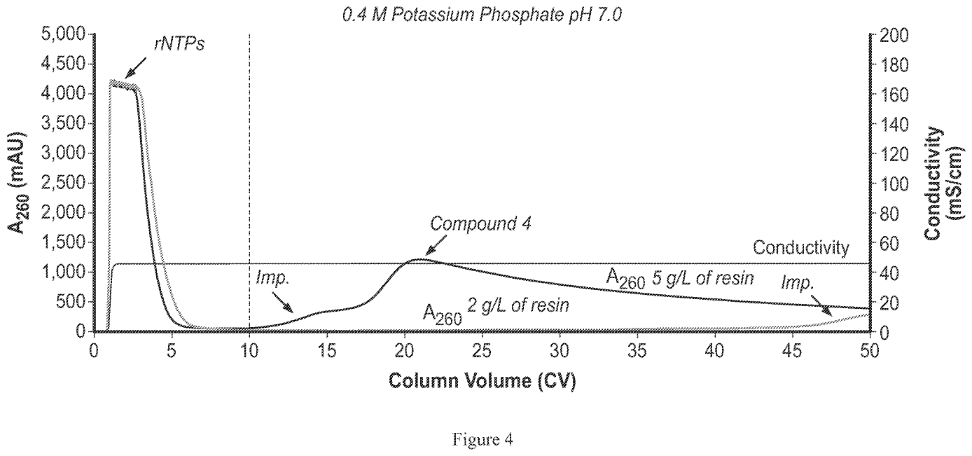

Residual rNTPs were removed using a 0.4 M potassium phosphate pH 7.0 wash for 10 CVs in all experimental conditions tested (see FIG. 4). Additionally, Compound 4 peaks eluting from the column at much later volume (>15 CVs, 5 g/L of resin).

### Example 6

### Evaluating the Impact of Feed Composition and Load Challenge

A mixture containing Compound 4 collected from an IVT preparation was loaded (2 or 5 g/L of resin) onto a 10 mL (1.0×12.7 cm) column packed with a low ionic capacity Nuvia aPrime 4A resin (87 μeq/mL). Additionally, a pump wash was performed prior to sample application in order to prime the system with load material and reduce the delay volume. After sample application onto the columns, the columns were washed with 0.4 M potassium phosphate pH 7.0 for 10 CVs. Columns were then washed with Milli-Q® water for 5 CVs and eluted with 0.4 M potassium chloride for 20 CVs. Peak fractions were collected once the absorbance at 260 nm was greater than 500 mAU. The columns were cleaned with 1 M NaOH for 5 CVs, neutralized in 1 M Tris-HCl pH 7.5 for 5 CVs, and rinsed in Milli-Q® water for 5 CVs.

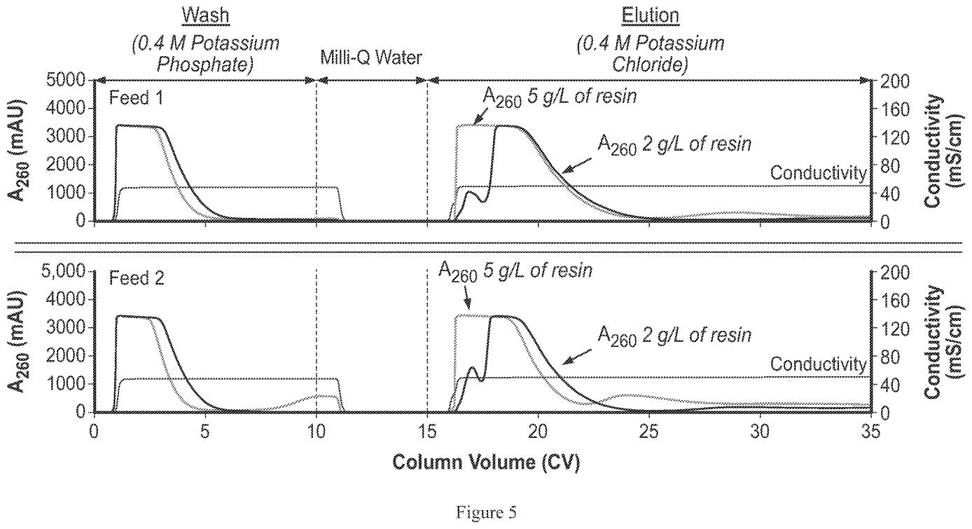

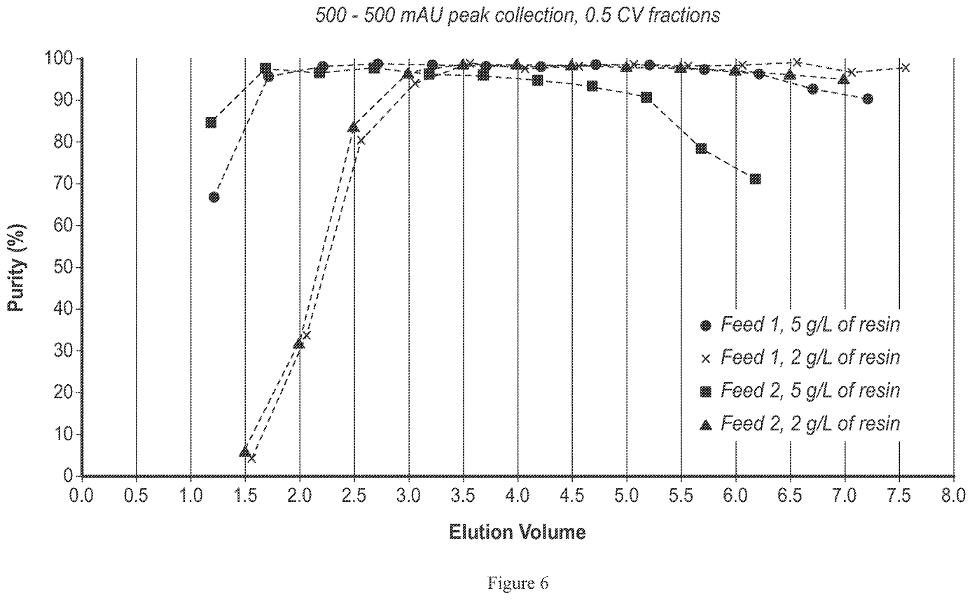

Feed material and load challenge influences Compound 4 peak migration during the wash and elution steps (see FIG. 5). Additionally, Compound 4 recovery and purity in each fraction collected varied based on the feed material and/or load challenge (2 or 5 g/L of resin) (see FIG. 6).

### Example 7

### Mixed-Mode Resin Screen Using De Novo Crude Reaction Mixture

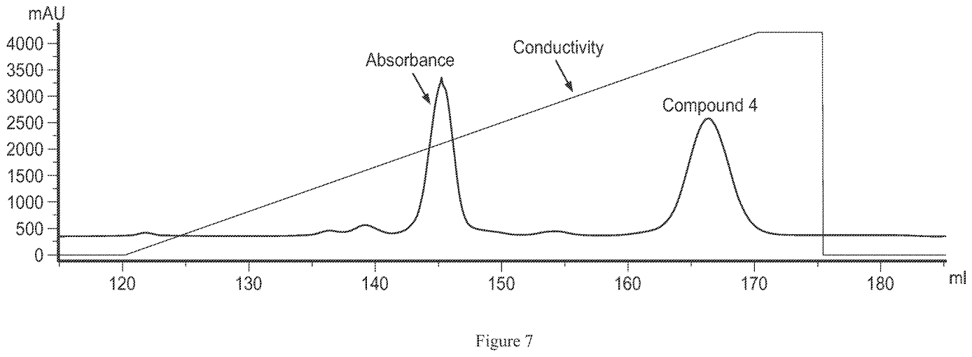

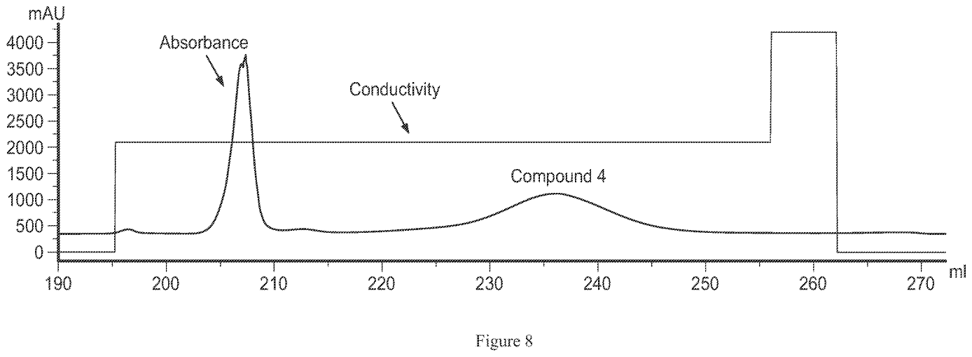

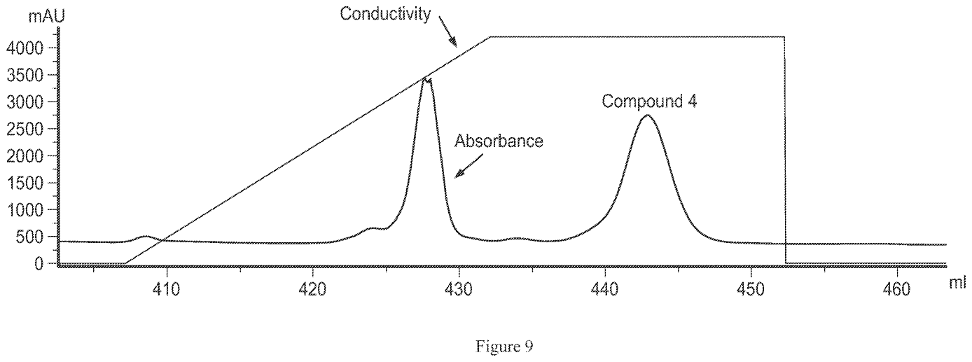

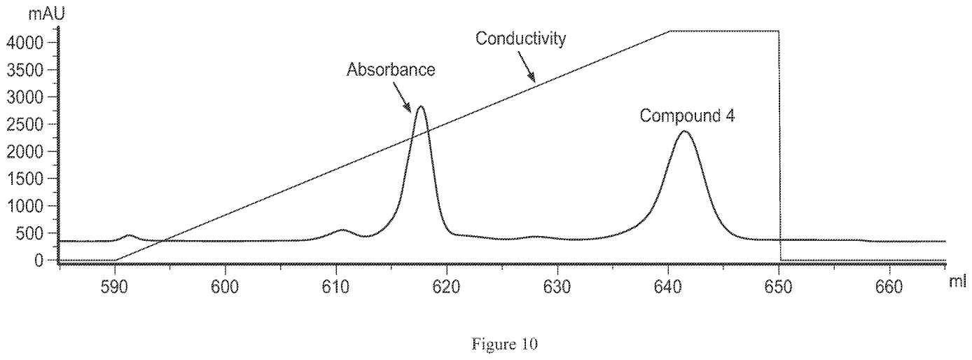

Crude reaction mixture from de novo synthesis of Compound 4 was loaded on to columns packed with Nuvia aPrime 4A with a load challenge of 1.8 mg/mL. The columns were washed with water for 1 CV. The contents of the columns were then eluted using the methods outlined in Table 7.1. Methods 1-4 provided separation between impurities and Compound 4 (see FIGS. 7-10, respectively).

TABLE 7.1
Elution Conditions

| | | Flow rate | |
|---|---|---|---|
| Method | Buffer | (mL/min) | Gradient |
| 1 | 0.75M | 2 | 1% Buffer to 100% Buffer in water in a |
| | NH₄Cl | | linear gradient for 10 CVs, 100% Buffer |
| | | | for 1 CV, and 100% water for 3 CVs |
| 2 | 0.75M | 2.5 | 50% Buffer 12 CVs, 100% Buffer for 1 |
| | NH₄Cl | | CV, and 100% water for 2 CVs |
| 3 | 0.75M | 2.5 | 1% Buffer to 100% Buffer in water in a |
| | KCl | | linear gradient for 5 CVs, 100% Buffer |
| | | | for 5 CVs, and 100% water for 2 CVs |
| 4 | 0.75M | 2.5 | 1% Buffer to 100% Buffer in water in a |
| | NaCl | | linear gradient for 10 CVs, 100% Buffer |
| | | | for 1 CV, and 100% water for 3 CVs |

### Example 8

### Evaluating Stability Compound 4

A mixture containing Compound 4 collected from an IVT preparation was loaded onto a column packed with Nuvia aPrime 4A resin. Ammonium chloride was used as the elution buffer (isocratic wash). The eluates from different cycles of the chromatography were pooled. The pooled fractions were then forward processed in tangential flow filtration (TFF), and the final material was exchanged into water (Pooled Eluate). The Pooled Eluate was aliquoted into two tubes. One was unadjusted (TFF Retentate) and the other was adjusted to pH 7.5 with a 1M Tris Buffer pH 7.5 (TFF Retentate pH 7.5). Each sample was kept at 5±3° C. The samples' purities were tested from initial week (TO) to 26 weeks (see to Table 8.1 for time points of the study).

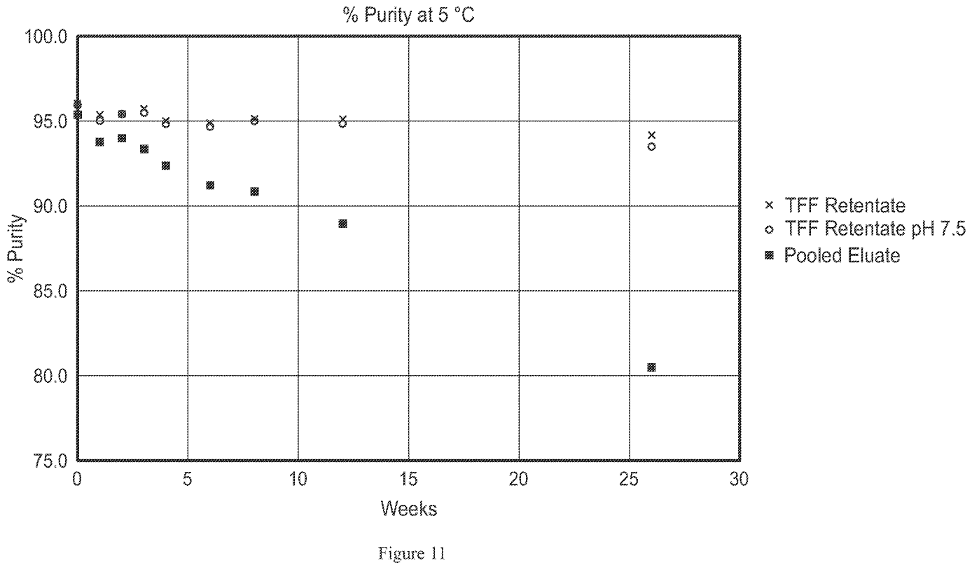

The purities of the TFF Retentate and TFF Retentate pH 7.5 were not affected up to 12 weeks. A drop in purity of 1.9% and 2.5% was seen for TFF retentate and TFF Retentate pH 7.5. However, Pooled Eluate observed a significant drop in purity over the 26 weeks of hold at 5±3° C. (see FIG. 11).

The study indicates that while the Pooled Eluate in ammonium chloride have a shorter hold time at 5±3° C. and should be processed within 2 weeks. Compound 4 after TFF (TFF Retentate and TFF Retentate pH 7.5) is stable in water for up to 6 months at 5±3° C.

TABLE 8.1
The experiment design with the timepoints present in this table
were followed to generate the data points for Compound 4 purity.

| Sample | Temperature (° C.) | Hold Time (wk.) |
|---|---|---|
| TFF Retentate | 5 ± 3 | 0 |
| | | 1 |
| | | 2 |
| | | 3 |
| | | 4 |
| | | 8 |
| | | 12 |
| | | 26 |
| TFF Retentate pH 7.5 | 5 ± 3 | 0 |
| | | 1 |
| | | 2 |
| | | 3 |
| | | 4 |
| | | 8 |
| | | 12 |
| | | 26 |
| Pooled Eluate | 5 ± 3 | 0 |
| | | 1 |
| | | 2 |
| | | 3 |
| | | 4 |
| | | 8 |
| | | 12 |
| | | 26 |

### Example 9

### One-Gram Scale Purification

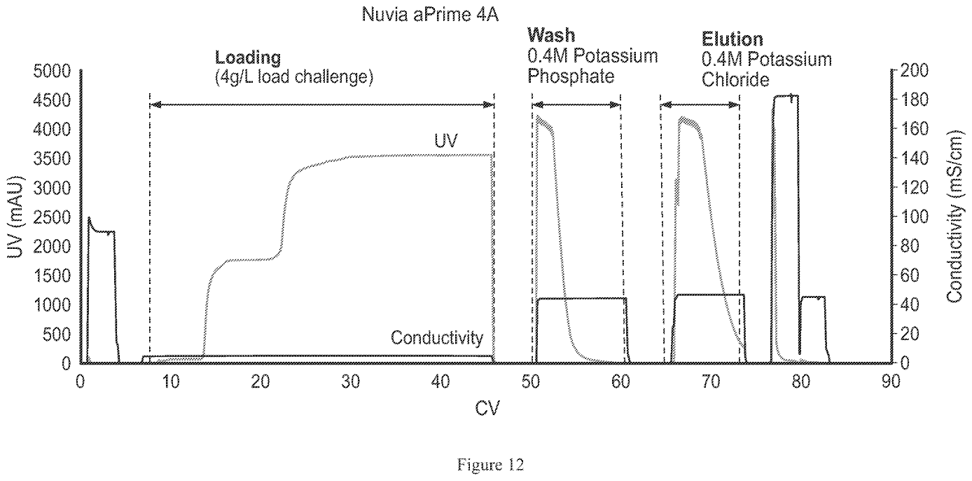

The load material was processed on a 294 mL Nuvia aPrime 4A column at load challenge of 4 g/L. The column was charged with 0.4 M potassium phosphate for 3 CVs. The equilibration was done in water and then the load material was added onto the column. The column was then wash with water followed by 0.4 M potassium phosphate. After washing the column with water again, the elution was done with 0.4 M potassium chloride (see FIG. 12). The eluate was collected in fractions, and each fraction was tested for purity. The fractions containing Compound 4 were pooled for a total purity of >95% of the pooled eluate. The pooled eluate was then processed on a 2 kDa (Sartocon® Hydrosart®) membrane. The total area of the TFF system was 0.06 m². This step was utilized to concentrate and to buffer exchange the pooled eluate into water (see Table 9.1 for summary of process parameter).

The final material was tested for concentration, purity, and recovery of each step. The process seemed to have generated material that was >95% at a concentration of 10 mM. The recovery from mixed-mode chromatography was about 90% and from TFF was 78%.

TABLE 9.1
The overall process parameter used during the experiment

| Unit Operation | Parameter | Value | Unit |
|---|---|---|---|
| Permeate | Volume | 10 | L |
| Storage | Temperature | 18 ± 3 | ° C. |
| Mixed-Mode | Resin | Nuvia aPrime 4A | |
| Chromatography | Column Volume | 0.294 | L |
| (Nuvia aPrime | Flow Rate | 0.062 | L/min |
| 4A) | Process Time | 5.5 | hr. |
| TFF | Membrane | Sartocon ® | |
| (2 kDa | | Hydrosart ® | |
| Sartocon ® | MWCO | 2 | kDa |
| Hydrosart ®) | Total Membrane Area | 0.06 | m² |
| | Feed Flow Rate | 0.3 | L/min. |
| | TMP | ≤35 | psig |
| | Process Time | 3 | hr. |

### Example 10

### Stability of the Mixed-Mode Chromatography Eluate for Intermediate Hold Times

The mixed-mode chromatography eluate generated in Example 9 with the new buffer matrix (0.4M potassium chloride) was analyzed for Compound 4 purity over a period of 14 days at 5±3° C. and room temperature (RT) (see Table 10.1 for the time points).

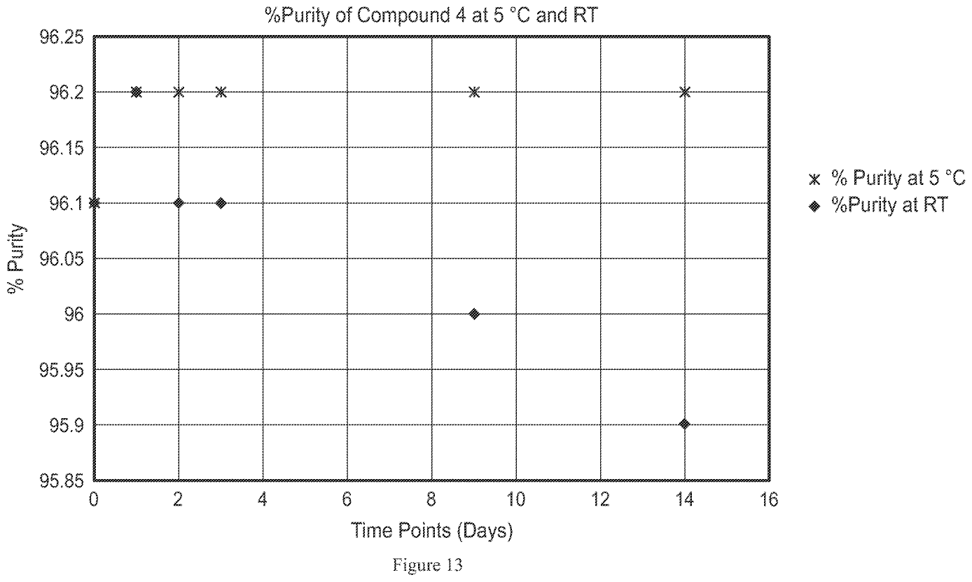

The Compound 4 stability drops significantly at room temperature (RT) in the 14 days hold time. However. Compound 4 is stable at 5±3° C. during the same 14 days hold time (see FIG. 13). The study concludes that the mixed-mode chromatography eluate can be stored up to 14 days in case of any processing holds without any purity concerns at 5±3° C.

TABLE 10.1
Timepoints plan for the hold time stability study

| Sample | Temperature (° C.) | Hold Time (Days) |
|---|---|---|
| Mixed-mode | 5 ± 3 | 0 |
| chromatography | | 1 |
| eluate | | 2 |
| | | 3 |
| | | 9 |
| | | 14 |
| | RT | 0 |
| | | 1 |
| | | 2 |
| | | 3 |
| | | 9 |
| | | 14 |

### Example 11

### Twenty-Gram Scale Purification

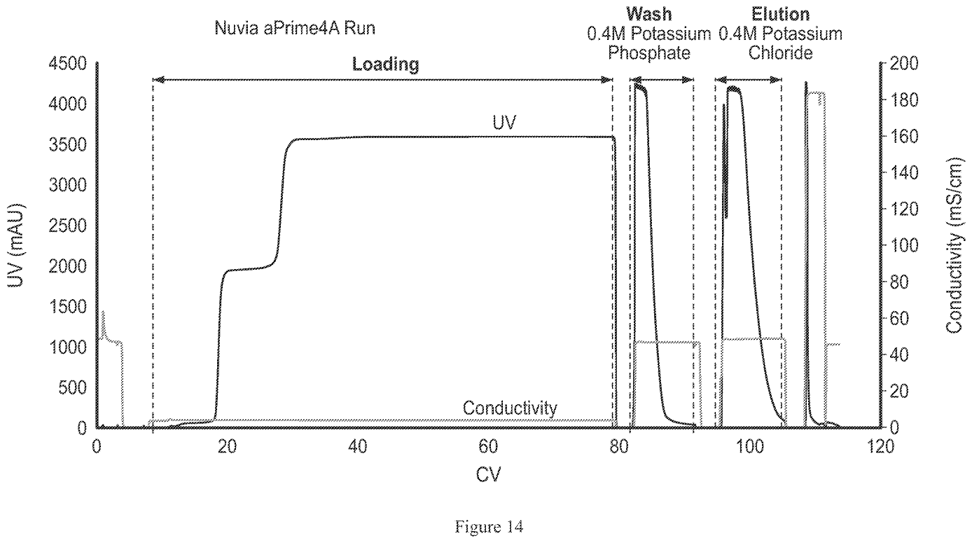

The total load was processed on a 980 mL Nuvia aPrime 4A column over 7 cycles with a load challenge of 4 g/L±1. The wash was 10 CVs of 0.4 M potassium phosphate and elution of 0.4 M potassium chloride (see FIG. 14). The elution phase was fractionated, and the fractions were analyzed for purity of Compound 4. The fractions were pooled to get a total purity of >90%. The pooled material was then forward processed on a Sartocon® Hydrosart® 2 kDa membrane in TFF1 with a total area of 1.2 m² to reach a concentration of 5 mM, and the resulting eluate was buffer exchanged into water for injection. The retentate from TFF1 was then processed on a smaller scale of Sartorius Hydrosart® 2 kDa membrane area of 0.1 m² (TFF2) that allowed for further concentrate of Compound 4 from 5 mM to 20 mM (see Table 11.1 for process parameters summary).

The final material (TFF2 retentate) was confirmed for identity of Compound 4 at a concentration of 20.3 mM. The UPLC analysis confirmed purity of 94.10. The final product was tested for sequence contamination by NGS since load material was pooled from different batches. The process showed robustness in deterring of any sequence contamination from the varied load material showcasing mixing of in-coming material to have no effect on the process.

TABLE 11.1
Summary of process parameters of a 20 gram batch cycle.

| Unit Operation | Parameter | Value | Unit |
|---|---|---|---|
| Permeate | Volume | 355 | L |
| Storage | Temperature | 18 ± 3 | ° C. |
| Mixed-Mode | Resin | Nuvia aPrime 4A | |
| Chromatography | Column Volume | 0.919 | L |
| (Nuvia aPrime | Flow Rate | 0.460 | L/min |
| 4A) | Process Time | 4 | hr. |
| TFF1 | Membrane | Sartocon ® | |
| (2 kDa | | Hydrosart ® | |
| Hydrosart ®) | MWCO | 2 | kDa |
| | Total Membrane Area | 1.2 | m² |
| | Feed Flow Rate | 6 | L/min. |
| | TMP | ≤35 | psig |
| | Process Time | 3 | hr. |
| TFF2 | Membrane | Sartocon Slice | |
| (2 kDa | | Hydrosart ® | |
| Hydrosart ®) | MWCO | 2 | kDa |
| | Total Membrane Area | 0.1 | m² |
| | Flow Rate | 2 | L/min. |
| | TMP | ≤35 | psig |
| | Process Time | 4 | hr. |
| | Waste Volume | 0.5 | L |

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

What is claimed is:

1. A method of purifying an oligonucleotide, or salt thereof, comprising:
collecting and combining one or more mixtures comprising the oligonucleotide, or a salt thereof, and one or more contaminants; and
removing the contaminants from the combined mixtures using a mixed-mode purification system.

2. The method of claim 1, wherein the oligonucleotide comprises 1 to 200, 1 to 175, 1 to 150, 1 to 125, 1 to 100, 1 to 75, 1 to 50, 1 to 25, 1 to 20, 1 to 15, 1 to 10, or 1 to 5 nucleotides.

3. The method of claim 1 or 2, wherein the oligonucleotide comprises 1 to 5 nucleotides.

4. The method of claim 1 or 2, wherein the oligonucleotide comprises 3 or 4 nucleotides.

5. The method of claim 1 or 2, wherein the oligonucleotide is an mRNA nucleotide cap.

6. The method of claim 1 or 2, wherein the oligonucleotide is a compound of Formula I: 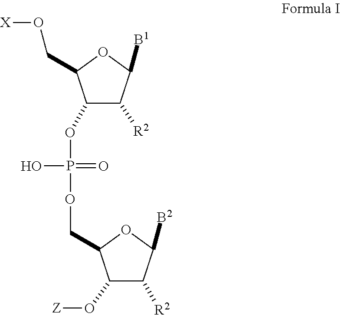
or salt thereof, wherein:
B¹ and B² are independently a natural, a modified, or an unnatural nucleoside based;
R¹ and R² are independently —OH or —OCH₃;
X is H or 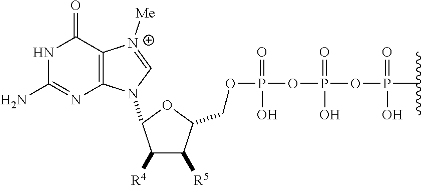
wherein R⁴ and R⁵ are independently —OH or —OCH₃; and
Z is H or 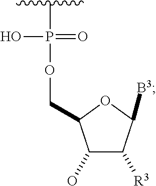
wherein B³ is a natural, a modified, or an unnatural nucleoside based; and
R³ is —OH or —OCH₃.

7. The method of claim 1 or 2, wherein the oligonucleotide is 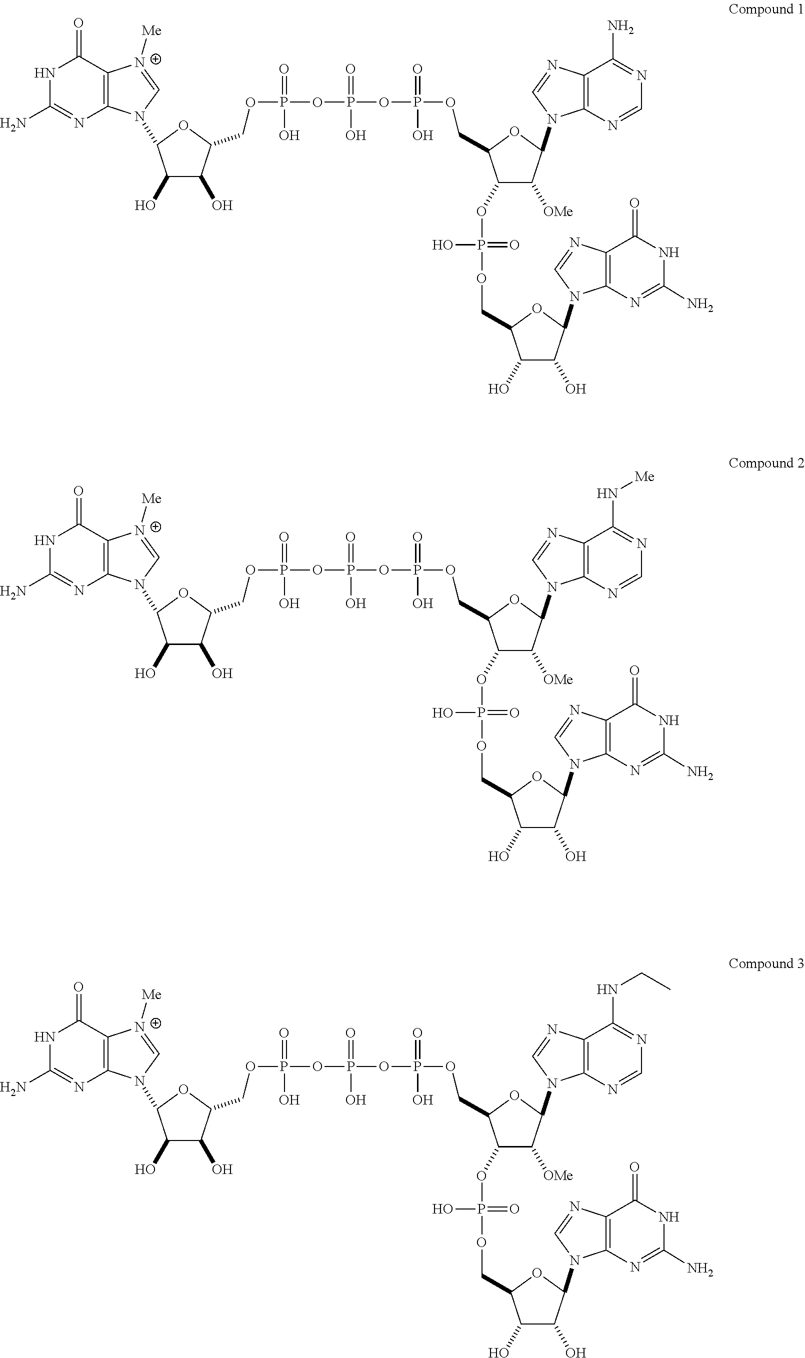 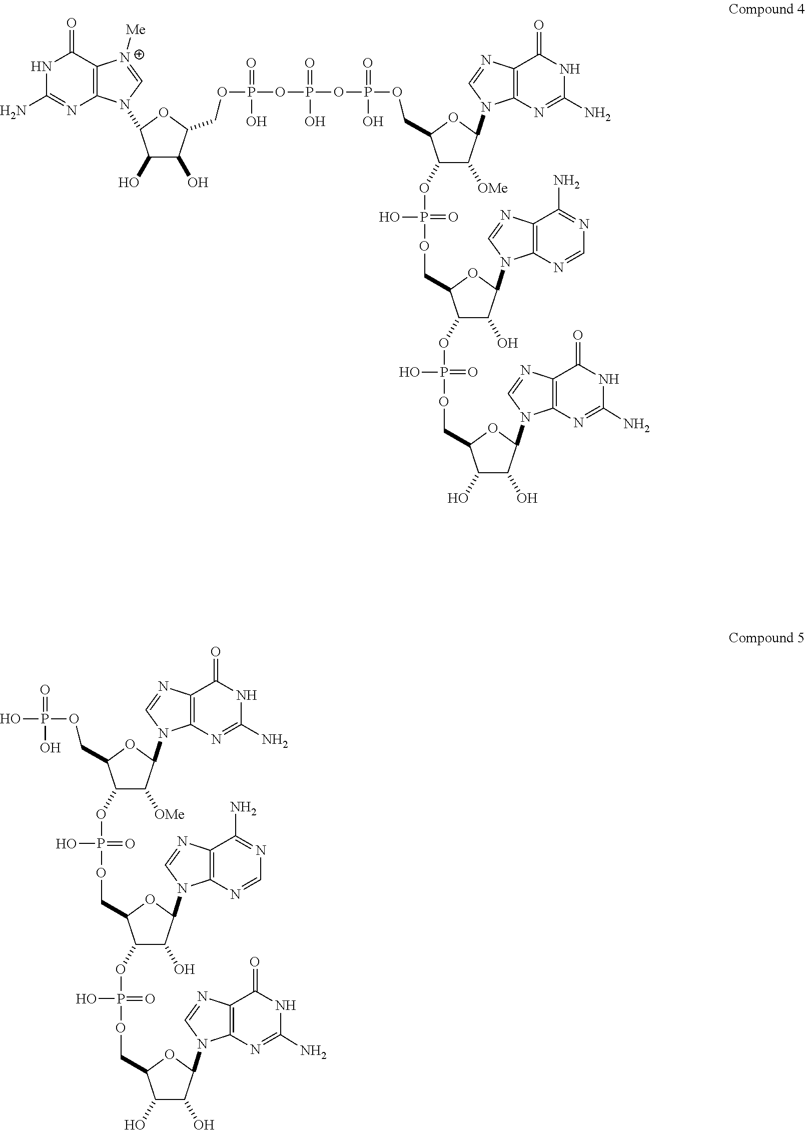
or a salt thereof.

8. The method of claim 1, 2, or 7, wherein the oligonucleotide is

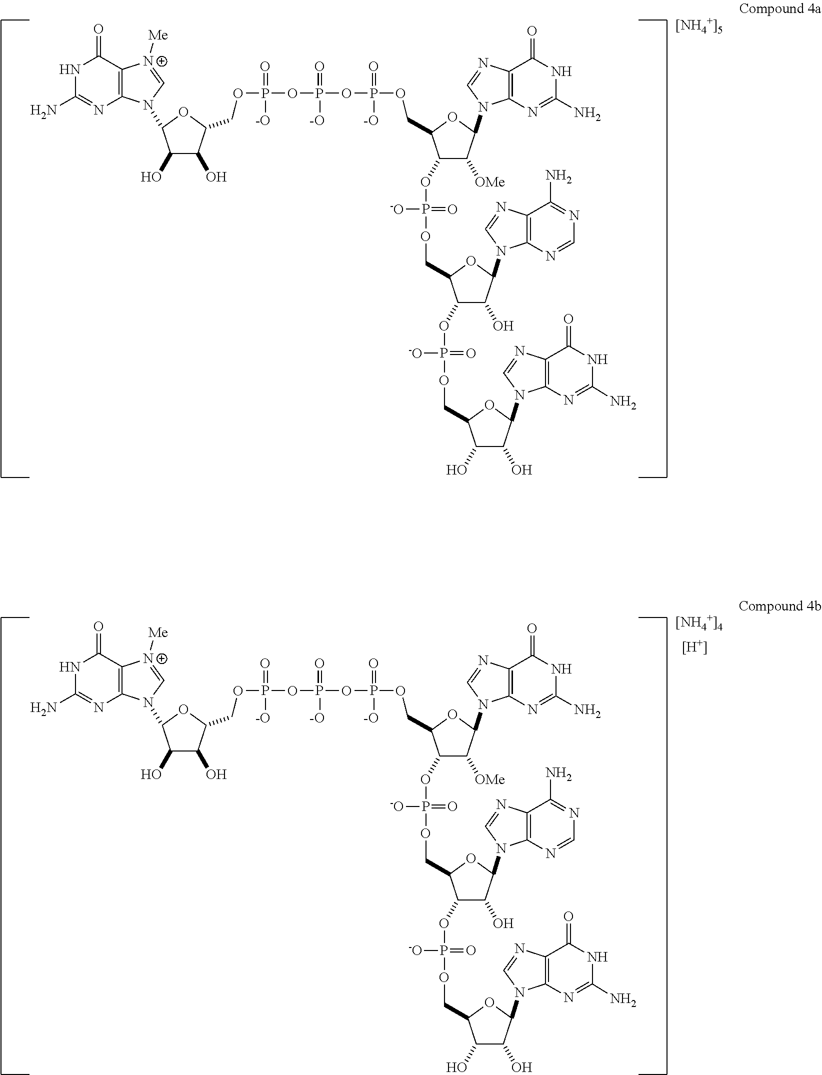

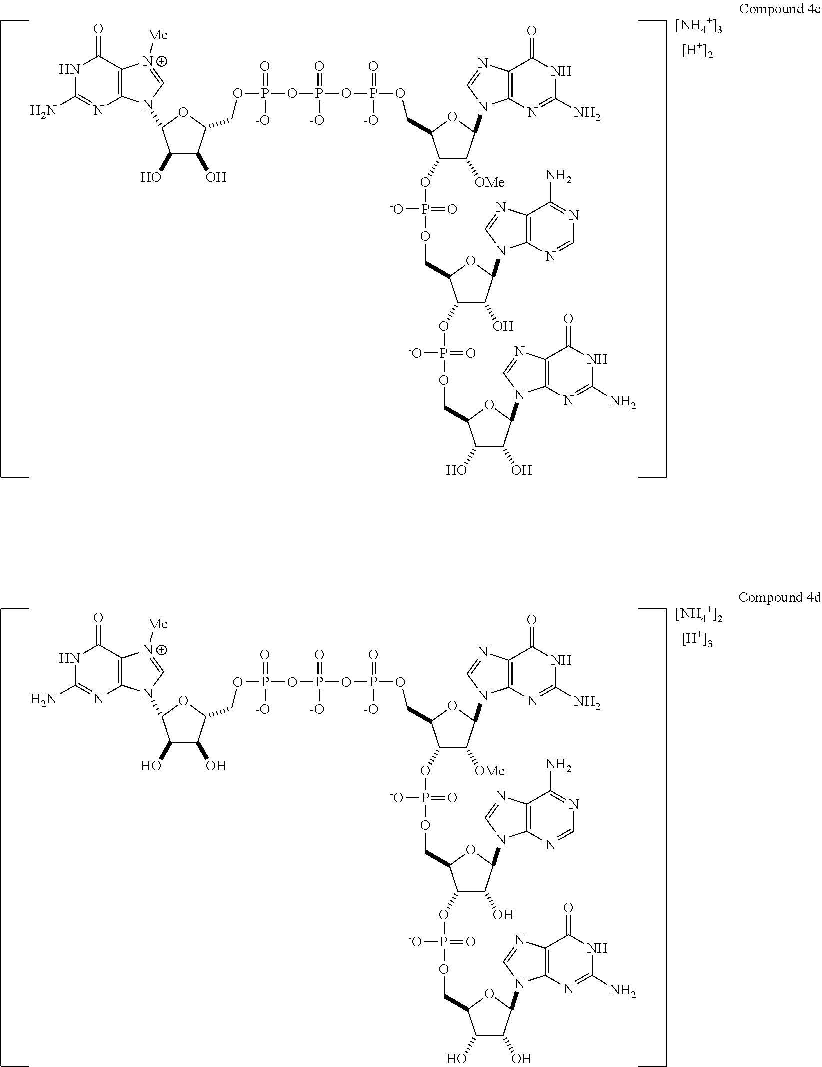

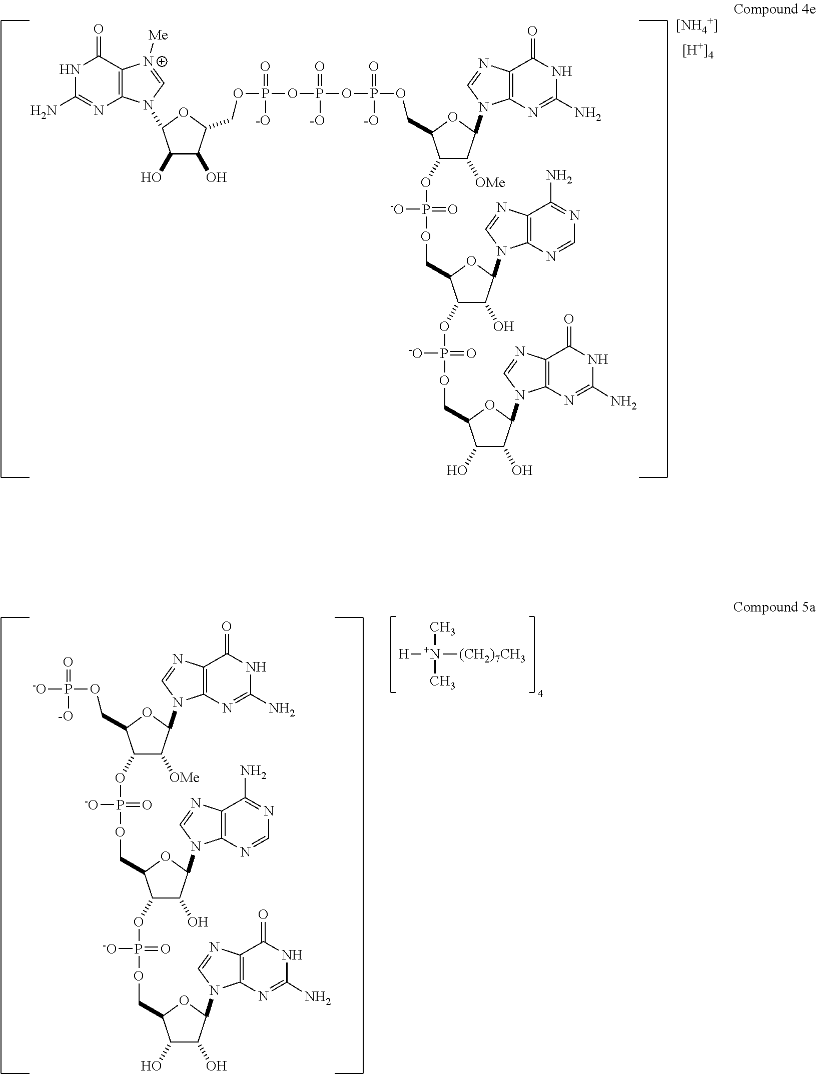

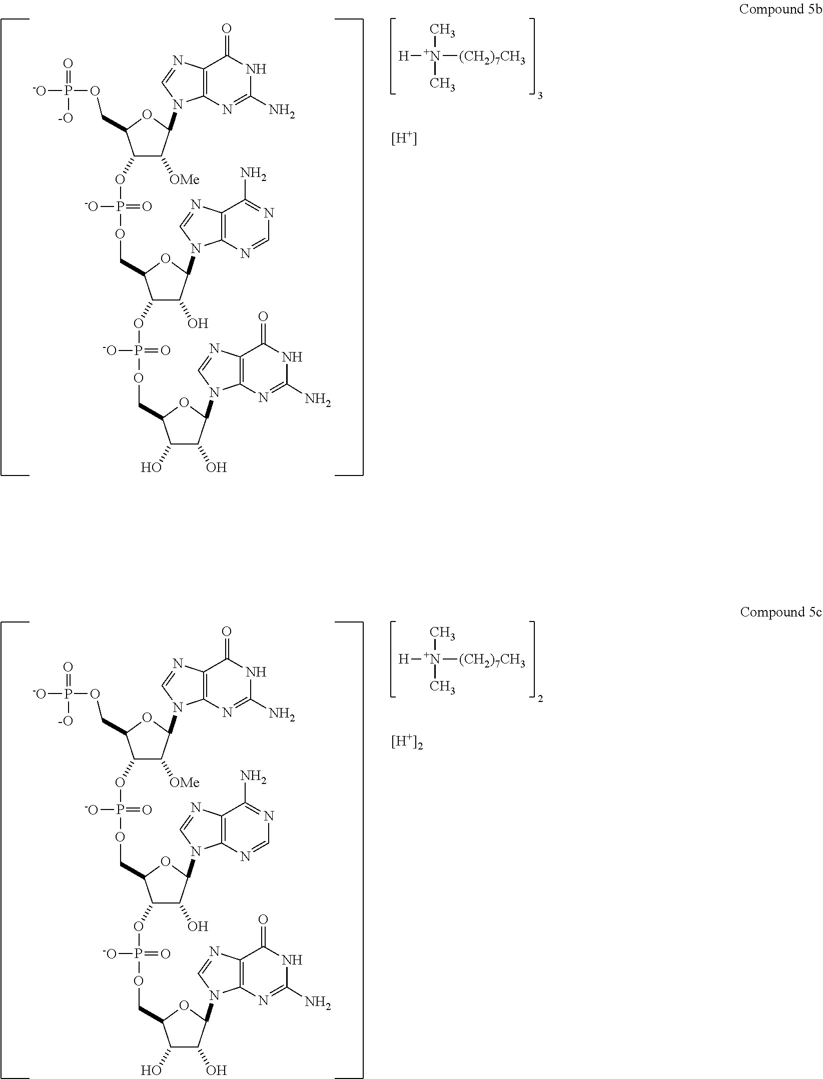

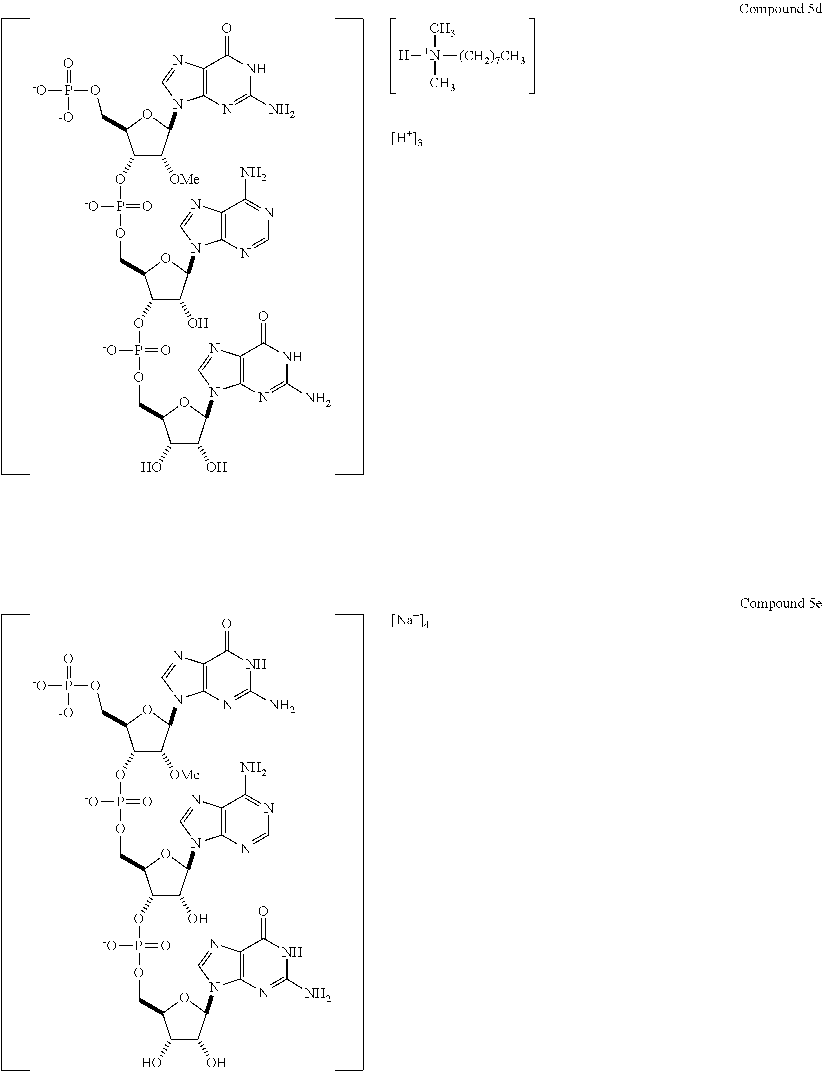

9. The method of any one of claims 1-8, wherein the one or more mixtures that are collected and combined are from an mRNA preparation.

10. The method of claim 9, wherein the mRNA preparation is an in vitro transcription preparation.

11. The method of any one of claims 1-8, wherein the one or more mixtures that are collected and combined from the purification of mRNA nucleotide caps that were prepared in a synthetic reaction mixtures.

12. The method of any one of claim 11, wherein the synthetic reaction mixture is a de novo preparation.

13. The method of any one of claims 1-12, wherein the one or more contaminants comprise macromolecules, proteins, or combinations thereof.

14. The method of any one of claims 1-13, wherein the one or more contaminants comprise ribonucleoside triphosphates (rNTPs).

15. The method of claim 14, wherein the rNTPs are rATP, rGTP, rCTP, rUTP, or m1Ψ, or combinations thereof.

16. The method of any one of claims 1-13, wherein the one or more contaminants comprise nucleic acid.

17. The method of claim 16, wherein the nucleic acid is RNA or DNA.

18. The method of claim 17, wherein the RNA is mRNA, tRNA, or rRNA.

19. The method of any one of claims 1-18, wherein the method of purifying the oligonucleotide results in the oligonucleotide with a purity of about 70% to about 100%, about 80% to about 100%, about 80% to about 99%, about 85% to about 99%, about 90% to about 99%, or about 95% to about 99%.

20. The method of any one of claims 1-19, wherein removing the contaminants from the combined mixtures using a mixed-mode purification system comprises passing the combined mixtures through a mixed-mode chromatography system to provide a first mixture.

21. The method of claim 20, wherein the mixed-mode chromatography system comprises a mixed-mode resin.

22. The method of claim 21, wherein the mixed-mode resin is a low ionic capacity resin.

23. The method of claim 21, wherein the mixed-mode resin is a high ionic capacity resin.

24. The method of any one of claims 21-23, wherein the mixed-mode resin comprises macroporous highly crosslinked polymer or high porosity cross-linked cellulose.

25. The method of any one of claims 21-23, wherein the mixed-mode resin comprises macroporous highly crosslinked polymer.

26. The method of any one of claims 21-25, wherein the mixed-mode resin comprises resins with hydrophobic and anion exchange properties.

27. The method of any one of claims 21-25, wherein the mixed-mode resin comprises a resin with hydrophobic and anion exchange properties.

28. The method of any one of claims 21-27, wherein the mixed-mode resin comprises an aromatic hydrophobic anion exchanger.

29. The method of claim 28, wherein the aromatic hydrophobic anion exchanger comprises a ligand of Formula II: 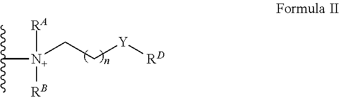
or a salt thereof, wherein:
R^{A} is —CH₃, —CH₂CH₃, —(CH₂)₂CH₃, —CH(CH₃)₂, and —(CH₂)₃CH₃;
R^{B} is —CH₃, —CH₂CH₃, —(CH₂)₂CH₃, —CH(CH₃)₂, and —(CH₂)₃CH₃;
n is 0, 1, 2, 3, or 4;
Y is absent, O, or NR^{C};
R^{C} is H or —CH₃; and
R^{D} is —CH₃, C₆₋₁₀ aryl, or 5 to 10 membered heteroaryl ring.

30. The method of claim 29, wherein the ligand is:

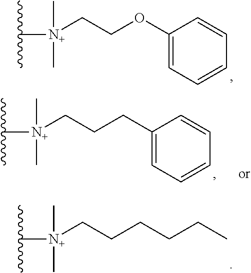

31. The method of claim 29, wherein the ligand is:

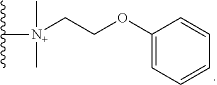 [SMILES: *[N+](C)(C)CCOc1ccccc1]

32. The method of claim 28, wherein the aromatic hydrophobic anion exchanger comprises a ligand having the formula:

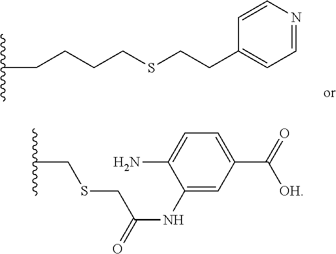 [SMILES: *CCCCSCCc1ccncc1 ; *CSCC(=O)Nc1cc(C(=O)O)ccc1N]

33. The method of any one of claims 21-25, wherein the mixed-mode resin comprises a resin with hydrophobic, anion exchange, and hydrogen bonding properties.

34. The method of claim 33, wherein the resin with hydrophobic, anion exchange, and hydrogen bonding properties comprises a ligand having the formula:

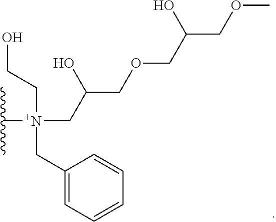 [SMILES: *[N+](CCO)(Cc1ccccc1)CC(O)COCC(O)COC]

35. The method of any one of claims 29-32 and 34, wherein the density of the ligands is 100±20 μeq/ml

36. The method of any one of claims 29-32 and 34, wherein the density of the ligands is (±20 μeq/ml) 50, 80, 100, 150, or 200 μeq/ml.

37. The method of any one of claims 21-25 wherein the mixed-mode resin comprises a resin with calcium affinity and cation exchange priorities.

38. The method of any one of claims 21-37, wherein the mixed-mode resin comprises particles with a median particle size of (±10 μm) 10 to 120, 20 to 100, 30 to 100, or 40 to 90 μm.

39. The method of any one of claims 21-37, wherein the mixed-mode resin comprises particles with a median particle size of (±10 m) 40, 50, 75, or 90 μm.

40. The method of any one of claims 21-37, wherein the mixed-mode resin comprises particles with a median particle size of 50±10 μm.

41. The method of claim 21, wherein the mixed-mode resin is Capto™ Adhere, Capto™ Adhere ImpRes, HEA HyperCel™, PPA HyperCel™, Nuvia aPrime 4A, MEP HyperCel, CMM HyperCel, CHT Ceramic Hydroxyapatite XT Media, CHT Ceramic Hydroxyapatite and Bio-Gel® Crystalline Hydroxyapatite, MPC Ceramic Hydroxyfluoroapatite Resin, or CFT Ceramic Fluoroapatite Resin.

42. The method of claim 21, wherein the mixed-mode resin is Capto™ Adhere, Capto™ Adhere ImpRes, HEA HyperCel™, PPA HyperCel™, or Nuvia aPrime 4A.

43. The method of any one of claims 21-23, wherein the mixed-mode resin is Nuvia aPrime 4A resin.

44. The method of any one of claims 20-43, wherein the mixed-mode chromatography system comprises about 0.08 to about 1.0 g/L of resin, about 0.2 to about 0.8 g/L of resin, or about 0.4 to about 0.6 g/L of resin.

45. The method of any one of claims 20-43, wherein the mixed-mode chromatography system comprises about 0.4 to about 8 g/L of resin, about 0.6 to about 6 g/L of resin, or about 0.8 to about 4 g/L of resin.

46. The method of any one of claims 20-43, wherein the mixed-mode chromatography system comprises 0.9 to about 11 g/L of resin, about 1 to about 9 g/L of resin, or about 3 to about 7 g/L of resin.

47. The method of any one of claims 20-43, wherein the mixed-mode chromatography system comprises about 0.5 g/L of resin, about 2 g/L of resin, or about 5 g/L resin.

48. The method of any one of claims 20-47, wherein the mixed-mode chromatography system comprises an about 3.5 to about 6.5 mL column, an about 4.0 to about 6.0 mL column, or an about 4.5 to about 5.5 mL column.

49. The method of any one of claims 20-47, wherein the mixed-mode chromatography system comprises an about 8.5 to about 11.5 mL column, an about 9.0 to about 11.0 mL column, or an about 9.5 to about 10.5 mL column.

50. The method of any one of claims 20-47, wherein the mixed-mode chromatography system comprises an about 5 mL column or an about 10 mL column.

51. The method of any one of claims 21-50, wherein the mixed-mode chromatography system comprises a first wash.

52. The method of claim 51, wherein the first wash is a first isocratic wash.

53. The method of claim 51, wherein the first wash is a first linear gradient wash.

54. The method of any one of claims 51-53, wherein the first wash comprises a first wash solution.

55. The method of claim 54, wherein the concentration of the first wash solution is about 0.1 M to about 0.7 M, about 0.2 M to about 0.6 M, or about 0.3 M to about 0.5 M.

56. The method of claim 54, wherein the concentration of the first wash solution is about 0.4 M to about 1.6 M, about 0.6 M to about 1.4 M, or about 0.8 M to about 1.2 M.

57. The method of any one of claims 51-56, wherein the first wash solution has a pH of about 6.7 to about 7.3, about 6.8 to about 7.2, or about 6.9 to about 7.1.

58. The method of any one of claims 51-57, wherein the first wash solution comprises a potassium phosphate solution.

59. The method of claim 58, wherein the concentration of the potassium phosphate solution is about 0.4 M or about 1.0 M.

60. The method of claim 58 or 59, wherein the potassium phosphate solution has a pH of about 6.7 to about 7.3, about 6.8 to about 7.2, or about 6.9 to about 7.1.

61. The method of claim 58 or 59, wherein the potassium phosphate solution has a pH of about 7.0.

62. The method of any one of claims 51-61, wherein the first wash is run for about 7 to about 12 column volumes (CVs), about 8 to about 11 CVs, or about 9 to about 10 CVs.

63. The method of any one of claims 51-61, wherein the first wash is run for about 10 CVs.

64. The method of any one of claims 51-63, wherein the first wash elutes bound contaminants.

65. The method of any one of claims 51-63, wherein the first wash elutes bound rNTP impurities.

66. The method of any one of claims 1-50, wherein the mixed-mode chromatography system does not comprises a first wash.

67. The method of any one of claims 1-66, wherein the mixed-mode chromatography system comprises a water wash.

68. The method of claim 67, wherein the water wash comprises Milli-Q® water.

69. The method of claim 67 or 68, wherein the first wash is run for about 3 to about 8 CVs, about 4 to about 7 CVs, or about 4 to about 6 CVs.

70. The method of claim 67 or 68, wherein the water wash is run for about 5 CVs.

71. The method of any one of claims 1-70, wherein the mixed-mode chromatography system comprises a second wash.

72. The method of claim 71, wherein the second wash is a second isocratic wash.

73. The method of claim 71, wherein the second wash is a second linear gradient wash.

74. The method of any one of claims 71-73, wherein the second wash comprises a second wash solution.

75. The method of claim 74, wherein the concentration of the second wash solution is about 0.1 M to about 0.7 M, about 0.2 M to about 0.6 M, or about 0.3 M to about 0.5 M.

76. The method of claim 74, wherein the concentration of the second wash solution is about 0.4 M to about 1.6 M, about 0.6 M to about 1.4 M, or about 0.8 M to about 1.2 M.

77. The method of claim 74, wherein the concentration of the second wash solution is about 0.25 M, about 0.5 M, about 0.75 M, or about 1.0 M.

78. The method of any one of claims 74-77, wherein the second wash solution has the same pH as the first wash solution.

79. The method of any one of claims 74-77, wherein the second wash solution has the same pH as the water wash.

80. The method of any one of claims 74-79, wherein the second wash solution has a pH of about 6.7 to about 7.3, about 6.8 to about 7.2, or about 6.9 to about 7.1.

81. The method of any one of claims 74-80, wherein the second wash solution comprises a potassium chloride solution, a sodium sulfate solution, a sodium citrate solution, a sodium chloride solution, or an ammonium chloride solution.

82. The method of any one of claims 74-80, wherein the second wash solution comprises a potassium chloride solution.

83. The method of claim 82, wherein the concentration of the potassium chloride solution is about 0.4 M or about 1.0 M.

84. The method of claim 82 or 83, wherein the potassium chloride solution has a pH of about 6.7 to about 7.3, about 6.8 to about 7.2, or about 6.9 to about 7.1.

85. The method of claim 82 or 83, wherein the potassium chloride solution has a pH of about 7.0.

86. The method of any one of claims 71-85, wherein the second wash is run for about 8 to about 14 CVs, about 9 to about 13 CVs, or about 10 to about 12 CVs.

87. The method of any one of claims 71-85, wherein the second wash is run for about 10 CVs or about 12 CVs.

88. The method of any one of claims 71-85, wherein the second wash is run for about 17 to about 25 CVs, about 18 to about 23 CVs, or about 19 to about 21 CVs.

89. The method of any one of claims 71-85, wherein the second wash is run for about 20 CVs.

90. The method of any one of claims 71-85, wherein the second wash eludes bound oligonucleotide.

91. The method of any one of claims 1-52, 54-65, 71, 72, and 74-90, wherein the mixed-mode chromatography system comprises a first isocratic wash and then a second isocratic wash.

92. The method of any one of claims 1-52, 54-65, 67-72, and 74-90, wherein the mixed-mode chromatography system comprises a first isocratic wash, then a water wash, and then a second isocratic wash.

93. The method of any one of claims 1-51, 53-65, 71, and 73-90, wherein the mixed-mode chromatography system comprises a first linear gradient wash and then a second linear gradient wash.

94. The method of any one of claims 1-51, 53-65, 67-71, and 73-90, wherein the mixed-mode chromatography system comprises a first linear gradient wash, then a water wash, and then a second linear gradient wash.

95. The method of any one of claims 1-50, 66, 71, and 73-90, wherein the mixed-mode chromatography system comprises a second linear gradient wash.

96. The method of any one of claims 1-50, 66, 71, 72, and 74-90, wherein the mixed-mode chromatography system comprises a second isocratic gradient wash.

97. The method of any one of claims 1-96 further comprising filtering the first mixture to provide a second mixture.

98. The method of claim 97, wherein the filtering the first mixture to provide a second mixture comprises a first tangential flow filtration.

99. The method of claim 98, wherein the first tangential flow filtration comprises a cassette filter, a spiral wound filter, a hollow fiber filter, a tubular filter, or a flat plate filter.

100. The method of claim 98, wherein the first tangential flow filtration comprises a cassette filter.

101. The method of any one of claims 98-100, wherein the first tangential flow filtration comprises a filter selected from a cellulose based membrane, a polyamide membrane, a polyethersulfone membrane, hydrophilic polyethersulfone membrane, polyvinylidene fluoride membrane, and a polyethylene membrane.

102. The method of any one of claims 98-100, wherein the first tangential flow filtration comprises a cellulose based membrane filter.

103. The method of claim 98, wherein the first tangential flow filtration comprises a Sartocon® Slice Hydrosart® membrane, Sartocon® Hydrosart® membrane, Sartorius Hydrosart® membrane, or Hydrosart® membrane.

104. The method of any one of claims 98-103, wherein the first tangential flow filtration comprises a filter having a total membrane area of about 2.1 to about 2.7 m², about 2.2 to about 2.6 m², or about 2.3 to about 2.5 m².

105. The method of any one of claims 98-103, wherein the first tangential flow filtration comprises a filter having a total membrane area of about 2.4 m².

106. The method of any one of claims 98-103, wherein the first tangential flow filtration comprises a filter having a total membrane area of about 0.1 to about 0.7 m², about 0.2 to about 0.6 m², or about 0.3 to about 0.5 m².

107. The method of any one of claims 98-103, wherein the first tangential flow filtration comprises a filter having a total membrane area of about 0.4 m².

108. The method of any one of claims 98-103, wherein the first tangential flow filtration comprises a filter having a total membrane area of about 0.6 to about 1.8 m², about 0.8 to about 1.6 m², or about 1.0 to about 1.4 m².

109. The method of any one of claims 98-103, wherein the first tangential flow filtration comprises a filter having a total membrane area of about 1.2 m².

110. The method of any one of claims 98-103, wherein the first tangential flow filtration comprises a filter having a total membrane area of about 0.05 to about 0.7 m², about 0.07 to about 0.5 m², or about 0.09 to about 0.3 m².

111. The method of any one of claims 98-103, wherein the first tangential flow filtration comprises a filter having a total membrane area of about 0.1 m².

112. The method of any one of claims 98-103, wherein the first tangential flow filtration comprises a filter having a total membrane area of about 0.03 to about 0.09 m², about 0.04 to about 0.08 m², or about 0.05 to about 0.07 m².

113. The method of any one of claims 98-103, wherein the first tangential flow filtration comprises a filter having a total membrane area of about 0.06 m².

114. The method of any one of claims 98-113, wherein the first tangential flow filtration comprises a filter having a molecular weight cut off of about 50 Da to about 5 kDa, about 100 Da to about 2 kDa, or about 250 Da to about 2 kDa.

115. The method of any one of claims 98-113, wherein the first tangential flow filtration comprises a filter having a molecular weight cut off of about 2 kDa.

116. The method of any one of claims 97-115, wherein the filtering the first mixture to provide a second mixture adjusts the concentration of the first mixture to about 14 mM to about 22 mM, about 16 mM to about 24 mM, or about 18 mM to about 22 mM.

117. The method of any one of claims 97-115, wherein the filtering the first mixture to provide a second mixture adjusts the concentration of the first mixture to about 19 mM, about 20 mM, or about 21 mM.

118. The method of any one of claims 97-117, wherein the filtering the first mixture to provide a second mixture comprises a first buffer exchange.

119. The method of claim 118, wherein the first buffer exchange comprises exchanging the solution of the first mixture to water for injection.

120. The method of any one of claims 97-117, wherein the filtering the first mixture to provide a second mixture does not comprise a first buffer exchange.

121. The method of any one of claims 1-120 further comprising filtering the second mixture to provide a third mixture.

122. The method of claim 121, wherein the filtering the second mixture to provide a third mixture comprises a second tangential flow filtration.

123. The method of claim 122, wherein the second tangential flow filtration comprises a cassette filter, a spiral wound filter, a hollow fiber filter, a tubular filter, or a flat plate filter.

124. The method of claim 122, wherein the second tangential flow filtration comprises a cassette filter.

125. The method of any one of claims 122-124, wherein the second tangential flow filtration comprises a filter selected from a cellulose based membrane, a polyamide membrane, a polyethersulfone membrane, hydrophilic polyethersulfone membrane, polyvinylidene fluoride membrane, and a polyethylene membrane.

126. The method of any one of claims 122-124, wherein the second tangential flow filtration comprises a cellulose based membrane filter.

127. The method of any one of claim 122, wherein the second tangential flow filtration comprises a Sartocon® Slice Hydrosart® membrane, Sartocon® Hydrosart® membrane, Sartorius Hydrosart® membrane, or Hydrosart® membrane.

128. The method of any one of claims 122-127, wherein the second tangential flow filtration comprises a filter having a total membrane area of about 2.1 to about 2.7 m², about 2.2 to about 2.6 m², or about 2.3 to about 2.5 m².

129. The method of any one of claims 122-127, wherein the second tangential flow filtration comprises a filter having a total membrane area of about 2.4 m².

130. The method of any one of claims 122-127, wherein the second tangential flow filtration comprises a filter having a total membrane area of about 0.1 to about 0.7 m², about 0.2 to about 0.6 m², or about 0.3 to about 0.5 m².

131. The method of any one of claims 122-127, wherein the second tangential flow filtration comprises a filter having a total membrane area of about 0.4 m², or.

132. The method of any one of claims 122-127, wherein the second tangential flow filtration comprises a filter having a total membrane area of about 0.6 to about 1.8 m2, about 0.8 to about 1.6 m², or about 1.0 to about 1.4 m².

133. The method of any one of claims 122-127, wherein the second tangential flow filtration comprises a filter having a total membrane area of about 1.2 m².

134. The method of any one of claims 122-127, wherein the second tangential flow filtration comprises a filter having a total membrane area of about 0.05 to about 0.7 m², about 0.07 to about 0.5 m², or about 0.09 to about 0.3 m².

135. The method of any one of claims 122-127, wherein the second tangential flow filtration comprises a filter having a total membrane area of about 0.1 m².

136. The method of any one of claims 122-127, wherein the second tangential flow filtration comprises a filter having a total membrane area of about 0.03 to about 0.09 m², about 0.04 to about 0.08 m², or about 0.05 to about 0.07 m².

137. The method of any one of claims 122-127, wherein the second tangential flow filtration comprises a filter having a total membrane area of about 0.06 m².

138. The method of any one of claims 122-137, wherein the second tangential flow filtration comprises a filter having a molecular weight cut off of about 50 Da to about 5 kDa, about 100 Da to about 2 kDa, or 250 Da to about 2 kDa.

139. The method of any one of claims 122-137, wherein the second tangential flow filtration comprises a filter having a molecular weight cut off of about 2 kDa.

140. The method of any one of claims 121-139, wherein the filtering the second mixture to provide a third mixture adjusts the concentration of the second mixture to about 14 mM to about 22 mM, about 16 mM to about 24 mM, or about 18 mM to about 22 mM.

141. The method of any one of claims 121-139, wherein the filtering the second mixture to provide a third mixture adjusts the concentration of the second mixture to about 19 mM, about 20 mM, or about 21 mM.

142. The method of any one of claims 121-141, wherein the filtering the second mixture to provide a third mixture the second mixture to provide a third mixture comprises a second buffer exchange.

143. The method of claim 142, wherein the second buffer exchange comprises exchanging the solution of the second mixture to water for injection.

144. The method of any one of claims 121-141, wherein the filtering the second mixture to provide a third mixture does not comprises a second buffer exchange.

145. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture; and
filtering the first mixture to provide a second mixture.

146. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture;
filtering the first mixture to provide a second mixture; and
filtering the second mixture to provide a third mixture.

147. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

148. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the filtering the second mixture to provide a third mixture comprises a second tangential flow filtration.

149. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash; and
filtering the first mixture to provide a second mixture, wherein the filtering the first mixture to provide a second mixture comprises first tangential flow filtration and a first buffer exchange.

150. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash; and
filtering the first mixture to provide a second mixture, wherein the filtering the first mixture to provide a second mixture comprises first tangential flow filtration and a first buffer exchange.

151. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

152. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration; and
filtering the second mixture to provide a third mixture, wherein the second mixture to provide a third mixture comprises a second tangential flow filtration and a second buffer exchange.

153. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

154. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration and a second buffer exchange.

155. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a first wash, a water wash, and a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

156. A method of purifying an oligonucleotide, or salt thereof, comprising:
collecting and combining one or more mixtures comprising the oligonucleotide, or a salt thereof, and one or more contaminants;
passing the combined mixtures through a mixed-mode chromatography system to provide a first mixture, wherein the mixed-mode chromatography system comprises a second wash;
filtering the first mixture to provide a second mixture, wherein the filtering the first mixture to provide a second mixture comprises a first tangential flow filtration and a first buffer exchange; and
filtering the second mixture to provide a third mixture, wherein the filtering the second mixture to provide a third mixture comprises a second tangential flow filtration.

157. An oligonucleotide purified according to a method in any one of claims 1-156.
